(19) United States
(12) Patent Application Publication
(10) Pub. No.: US 2021/0387990 A1
(43) Pub. Date: Dec. 16, 2021

(54) METHOD FOR PRODUCING DIMETHOXYBENZENE COMPOUND

(71) Applicant: TAIHO PHARMACEUTICAL CO., LTD., Tokyo (JP)

(72) Inventor: Masashi KONDO, Saitama (JP)

(73) Assignee: TAIHO PHARMACEUTICAL CO., LTD., Tokyo (JP)

(21) Appl. No.: 17/292,289

(22) PCT Filed: Nov. 8, 2019
(86) PCT No.: PCT/JP2019/043857

§ 371 Date: May 7, 2021

(30) Foreign Application Priority Data
Nov. 9, 2018 (JP) PCT/JP2018/041744
Mar. 11, 2019 (JP) 2019-044236

(51) Int. Cl. C07D 487/04

(52) U.S. Cl. CPC C07D 487/04

(57) ABSTRACT

A method for producing a compound represented by formula (A-1) or a salt thereof, the method comprising reacting (S)-3-((3,5-dimethoxyphenyl)ethynyl)-1-(pyrrolidin-3-yl)-1H-pyrazolo[3,4-d]pyrimidin-4-amine or a salt thereof with a compound represented by formula (I-1-A). Formula (I-1-A) and formula (A-1) are as described in the specification.

### CROSS-REFERENCE TO RELATED PATENT APPLICATIONS

The present application is a 35 U.S.C. 371 National Phase application from PCT/JP2019/043857 filed Nov. 8, 2019, which claims priority to international application number PCT/JP2018/041744, filed on Nov. 9, 2018, and Japanese Patent Application No. 2019-044236, filed on Mar. 11, 2019, the entire disclosures of which are hereby incorporated by reference.

### TECHNICAL FIELD

### Background Art

Acrylamide is one type of amide and is composed of acrylic acid and an amine. Acrylamide is industrially produced by hydrating acrylonitrile and is mainly used in starting materials for polyacrylamide. A typical experimental method for producing acrylamide allows acryloyl chloride or acrylic anhydride to act on a target amine to produce acrylamide.

Some acrylamide derivatives are known to be a compound that has an antitumor effect, and literature discloses that acrylamide derivatives can be induced in accordance with the above method. Additionally, a method for synthesizing a compound by using 3-chloropropionyl chloride instead of acryloyl chloride or acrylic anhydride is also disclosed (PTL 1 to 3).

The compound (S)-1-(3-(4-amino-3-((3,5-dimethoxyphenyl)ethynyl)-1H-pyrazolo[3,4-d]pyrimidin-1-yl)pyrrolidin-1-yl)-2-propen-1-one (“compound A” below in this specification) is reported as having excellent inhibitory activity on the fibroblast growth factor receptor (FGFR) and exhibiting antitumor activity (PTL 4 to 8). A method for synthesizing compound A by using (S)-3-((3,5-dimethoxyphenyl)ethynyl)-1-(pyrrolidin-3-yl)-1H-pyrazolo[3,4-d]pyrimidin-4-amine (“compound B” in this specification) and acryloyl chloride has been disclosed (PTL 4). It has also been reported that compound A can be obtained in the form of crystals by using various solvents (PTL 8).

### CITATION LIST

### Patent Literature

PTL 1: WO2016/115356A

PTL 2: JP2018-002662A

PTL 3: Chinese Patent No. 105859721B

PTL 4: WO2013/108809A

PTL 5: WO2015/008844A

PTL 6: WO2015/008839A

PTL 7: WO2016/159327A

PTL 8: WO2017/150725A

### SUMMARY OF INVENTION

### Technical Problem

Compound A has excellent FGFR inhibition activity and antitumor activity. Thus, an object of the present invention is to provide a method for producing compound A or a pharmaceutically acceptable salt thereof that is capable of mass synthesis, is simple and excellent in ease of use, and satisfies the quality of compound A or a pharmaceutically acceptable salt thereof required as a pharmaceutical product.

### Solution to Problem

The present inventor conducted extensive research to find a production method that is excellent in ease of use, including fewer operation steps in mass synthesis, and is capable of producing compound A or a pharmaceutically acceptable salt thereof in a short time, while maintaining the quality of compound A or a pharmaceutically acceptable salt thereof required as a pharmaceutical product. However, a problem was found that (S)—N-(1-(1-acryloylpyrrolidin-3-yl)-3-((3,5-dimethoxyphenyl) ethynyl)-1H-pyrazolo[3,4-d]pyrimidin-4-yl)acrylamide (“diamide compound” below) shown below contaminates compound A.

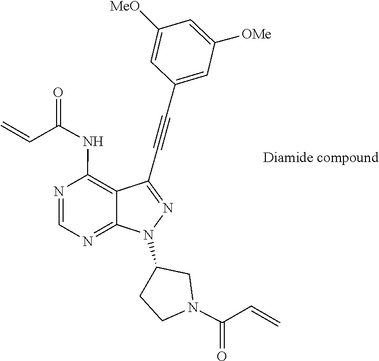

wherein Me represents a methyl group. The same applies below in the present specification.

The inventor conducted extensive research on reaction conditions for the method for producing compound A by using acryloyl chloride (PTL 4) in order to remove the diamide compound or suppress the formation of the diamide compound. However, the method for reducing the equivalent amount of acryloyl chloride was expected to leave compound B, thus decreasing the yield. A method for decomposing the diamide also led to a decrease in the yield of compound A and required a long time for filtering compound A in crystallization. Although the inventor further examined crystallization conditions, he had difficulty in efficiently removing the diamide compound. Thus, for these reasons, he found it difficult to mass-produce compound A while maintaining the quality of compound A required as a pharmaceutical product by the method for producing compound A by using acryloyl chloride.

The inventor further conducted extensive research and found a method for producing compound A or a pharmaceutically acceptable salt thereof that suppresses the formation of the diamide compound and is capable of mass production of compound A or a pharmaceutically acceptable salt thereof that satisfies the quality required as a pharmaceutical product by replacing acryloyl chloride by a specific acryloylating reagent.

Specifically, the present invention includes the following [1] to [27].

[1] A method for producing a compound represented by the following formula (A-1) or a salt thereof, the method comprising reacting 1 equivalent of (S)-3-((3,5-dimethoxyphenyl)ethynyl)-1-(pyrrolidin-3-yl)-1H-pyrazolo[3,4-d]pyrimidin-4-amine or a salt thereof with 1.0 to 1.3 equivalents of a compound represented by the following formula (I-1-A)

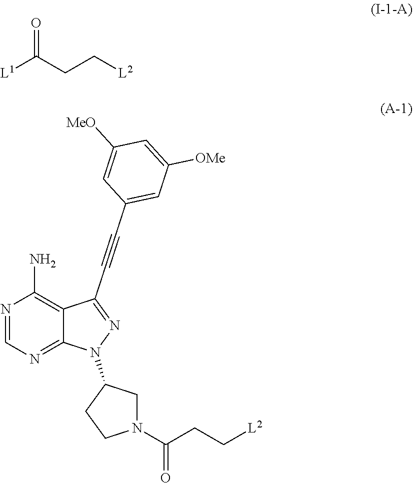

wherein L¹ and L² are the same or different, and each represents a leaving group.
[2] The method according to [1], wherein L¹ and L² are the same or different, and each is a halogen atom.
[3] The method according to [1], wherein the compound represented by formula (I-1-A) is 3-chloropropionyl chloride.
[4] The method according to [1], wherein the reaction is performed in the presence of at least one base selected from the group consisting of organic amine bases and inorganic bases.
[5] The method according to [4], wherein the base is a base containing a hydroxide ion.
[6] The method according to [4], wherein the amount of the base after subtracting an equivalent amount neutralized with an acid addition salt of (S)-3-((3,5-dimethoxyphenyl)ethynyl)-1-(pyrrolidin-3-yl)-1H-pyrazolo[3,4-d]pyrimidin-4-amine or a salt thereof is 0.5 to 10 equivalents.
[7] A method for producing (S)-1-(3-(4-amino-3-((3,5-dimethoxyphenyl)ethynyl)-1H-pyrazolo[3,4-d]pyrimidin-1-yl)pyrrolidin-1-yl)-2-propen-1-one or a pharmaceutically acceptable salt thereof, the method comprising:

producing a compound represented by formula (A-1) or a salt thereof by the method of [1]; and

eliminating L² from the obtained compound represented by formula (A-1).

[8] The method according to [7], wherein in the step of producing a compound represented by formula (A-1) or a salt thereof, a reaction is performed in the presence of at least one base selected from the group consisting of organic amine bases and inorganic bases.
[9] The method according to [8], wherein the base is a base containing a hydroxide ion.
[10] The method according to claim [8], wherein the amount of the base after subtracting an equivalent amount neutralized with an acid addition salt of (S)-3-((3,5-dimethoxyphenyl)ethynyl)-1-(pyrrolidin-3-yl)-1H-pyrazolo[3,4-d]pyrimidin-4-amine or a salt thereof is 0.5 to 10 equivalents per equivalent of (S)-3-((3,5-dimethoxyphenyl)ethynyl)-1-(pyrrolidin-3-yl)-1H-pyrazolo[3,4-d]pyrimidin-4-amine.
[11] The method according to claim [1] for improving the yield of (S)-1-(3-(4-amino-3-((3,5-dimethoxyphenyl)ethynyl)-1H-pyrazolo[3,4-d]pyrimidin-1-yl)pyrrolidin-1-yl)-2-propen-1-one or a pharmaceutically acceptable salt thereof.
[12] The method according to [11], wherein the yield of (S)-1-(3-(4-amino-3-((3,5-dimethoxyphenyl)ethynyl)-1H-pyrazolo[3,4-d]pyrimidin-1-yl)pyrrolidin-1-yl)-2-propen-1-one or a pharmaceutically acceptable salt thereof is improved by suppressing the formation of a diamide compound.
[13] A method for improving filterability, the method comprising producing (S)-1-(3-(4-amino-3-((3,5-dimethoxyphenyl)ethynyl)-1H-pyrazolo[3,4-d]pyrimidin-1-yl)pyrrolidin-1-yl)-2-propen-1-one or a salt thereof by the method of [1].
[14] A method for improving the yield of (S)-1-(3-(4-amino-3-((3,5-dimethoxyphenyl)ethynyl)-1H-pyrazolo[3,4-d]pyrimidin-1-yl)pyrrolidin-1-yl)-2-propen-1-one or a pharmaceutically acceptable salt thereof, the method comprising producing (S)-1-(3-(4-amino-3-((3,5-dimethoxyphenyl)ethynyl)-1H-pyrazolo[3,4-d]pyrimidin-1-yl)pyrrolidin-1-yl)-2-propen-1-one or a salt thereof by the method of [1].
[15] A method for producing a compound represented by the following formula (A-1) or a salt thereof, the method comprising reacting (S)-3-((3,5-dimethoxyphenyl)ethynyl)-1-(pyrrolidin-3-yl)-1H-pyrazolo[3,4-d]pyrimidin-4-amine or a salt thereof with a compound represented by the following formula (I-1-A) in the presence of a base containing a hydroxide ion

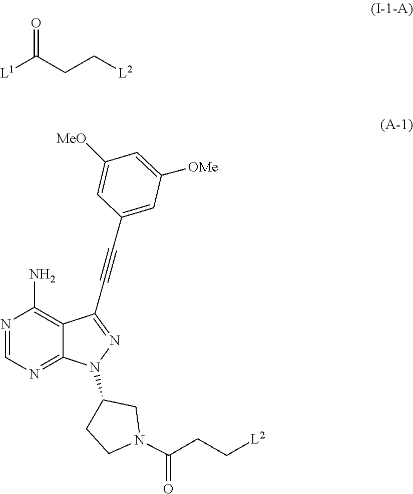

wherein L¹ and L² are the same or different, and each represents a leaving group.
[16] The method according to [15], wherein L¹ and L² are the same or different, and each is a halogen atom.
[17] The method according to [15], wherein the compound represented by formula (I-1-A) is 3-chloropropionyl chloride.
[18] The method according to [15], wherein the amount of the base after subtracting an equivalent amount neutralized with an acid addition salt of (S)-3-((3,5-dimethoxyphenyl)ethynyl)-1-(pyrrolidin-3-yl)-1H-pyrazolo[3,4-d]pyrimidin-4-amine or a salt thereof is 0.5 to 10 equivalents per equivalent of (S)-3-((3,5-dimethoxyphenyl)ethynyl)-1-(pyrrolidin-3-yl)-1H-pyrazolo[3,4-d]pyrimidin-4-amine.
[19] A method for producing (S)-1-(3-(4-amino-3-((3,5-dimethoxyphenyl)ethynyl)-1H-pyrazolo[3,4-d]pyrimidin-1-yl)pyrrolidin-1-yl)-2-propen-1-one or a pharmaceutically acceptable salt thereof, the method comprising:

producing a compound represented by formula (A-1) or a salt thereof by the method of [15]; and

eliminating L² from the obtained compound represented by formula (A-1).

[20] The method according to [19] for improving the yield of (S)-1-(3-(4-amino-3-((3,5-dimethoxyphenyl)ethynyl)-1H-pyrazolo[3,4-d]pyrimidin-1-yl)pyrrolidin-1-yl)-2-propen-1-one or a pharmaceutically acceptable salt thereof.
[21] The method according to [19], wherein the yield of (S)-1-(3-(4-amino-3-((3,5-dimethoxyphenyl)ethynyl)-1H-pyrazolo[3,4-d]pyrimidin-1-yl)pyrrolidin-1-yl)-2-propen-1-one or a pharmaceutically acceptable salt thereof is improved by suppressing the formation of a diamide compound.
[22] A method for improving filterability, the method comprising producing (S)-1-(3-(4-amino-3-((3,5-dimethoxyphenyl)ethynyl)-1H-pyrazolo[3,4-d]pyrimidin-1-yl)pyrrolidin-1-yl)-2-propen-1-one or a salt thereof by the method of [15].
[23] A method for improving the yield of (S)-1-(3-(4-amino-3-((3,5-dimethoxyphenyl)ethynyl)-1H-pyrazolo[3,4-d]pyrimidin-1-yl)pyrrolidin-1-yl)-2-propen-1-one or a pharmaceutically acceptable salt thereof, the method comprising producing (S)-1-(3-(4-amino-3-((3,5-dimethoxyphenyl)ethynyl)-1H-pyrazolo[3,4-d]pyrimidin-1-yl)pyrrolidin-1-yl)-2-propen-1-one or a salt thereof by the method of [15].
[24] A compound represented by the following formula (A-1) or a salt thereof

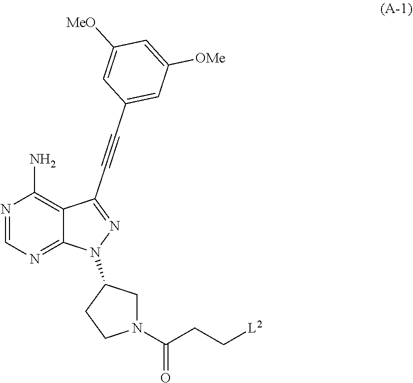

wherein L² represents a leaving group.
[25] The compound or a salt thereof according to [24], wherein L² is a halogen atom.
[26] The compound or a salt thereof according to [24], wherein L² is a chlorine atom.
[27] The compound or a salt thereof according to [24] for use in determining the presence of impurities in the production of (S)-1-(3-(4-amino-3-((3,5-dimethoxyphenyl)ethynyl)-1H-pyrazolo[3,4-d]pyrimidin-1-yl)pyrrolidin-1-yl)-2-propen-1-one or a pharmaceutically acceptable salt thereof.

### Advantageous Effects of Invention

The present invention provides a method for producing compound A or a pharmaceutically acceptable salt thereof that reduces the formation of related substances of compound A with excellent yield and excellent ease of use in mass production by using compound B or a salt thereof and a specific acryloylating reagent.

### DESCRIPTION OF EMBODIMENTS

In the present invention, compound A is (S)-1-(3-(4-amino-3-((3,5-dimethoxyphenyl)ethynyl)-1H-pyrazolo[3,4-d]pyrimidin-1-yl)pyrrolidin-1-yl)-2-propen-1-one. The structure of compound A is shown below.

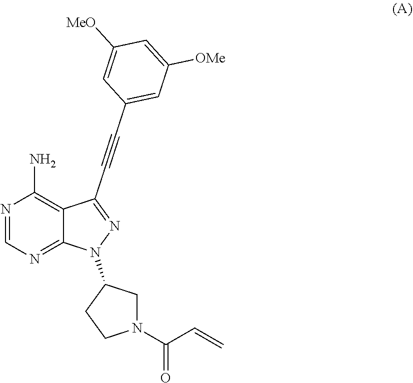 [SMILES: C=CC(=O)N1CC[C@H](n2nc(C#Cc3cc(OC)cc(OC)c3)c3c(N)ncnc32)C1]

Compound A or a pharmaceutically acceptable salt thereof may be a solvate (e.g., a hydrate) or a non-solvate. In the present invention, any of such forms are included within the scope of “compound A or a pharmaceutically acceptable salt thereof.” The pharmaceutically acceptable salt of compound A is not particularly limited, and examples include addition salts with inorganic acids such as hydrochloric acid and sulfuric acid; addition salts with organic acids such as acetic acid, citric acid, tartaric acid, and maleic acid; salts with alkali metals such as potassium and sodium; salts with alkaline earth metals such as calcium and magnesium; salts with organic bases, such as ammonium salts, ethylamine salts, and arginine salts; and the like. In the present specification, the term “compound A” may be intended to include a pharmaceutically acceptable “salt” and a “solvate” of compound A.

In the present invention, compound B is (S)-3-((3,5-dimethoxyphenyl)ethynyl)-1-(pyrrolidin-3-yl)-1H-pyrazolo[3,4-d]pyrimidin-4-amine. The structure of compound B is shown below.

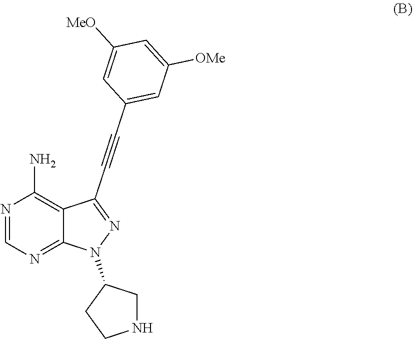 [SMILES: COc1cc(C#Cc2nn([C@H]3CCNC3)c3ncnc(N)c23)cc(OC)c1]

Compound B or a salt thereof may be a solvate (e.g., a hydrate) or a non-solvate. In the present invention, any of such forms are included within the scope of “compound B or a salt thereof.” The salt of compound B is not particularly limited, and examples include addition salts with inorganic acids such as hydrochloric acid, hydrobromic acid, hydroiodic acid, hydrofluoric acid, and sulfuric acid; addition salts with alkyl sulfuric acids such as methanesulfonic acid, p-toluenesulfonic acid, and benzenesulfonic acid; addition salts with organic acids such as acetic acid, citric acid, tartaric acid, and maleic acid; salts with alkali metals such as potassium and sodium; salts with alkaline earth metals such as calcium and magnesium; salts with organic bases, such as ammonium salts, ethylamine salts, and arginine salts; and the like. In the present specification, the term “compound B” may be intended to include a “salt” and “solvate” of compound B.

In the present invention, compound B used for producing compound A may be in free or salt form and is preferably an addition salt with an inorganic acid, an alkyl sulfuric acid, or an organic acid, more preferably an addition salt with an alkyl sulfuric acid, and even more preferably an addition salt with methanesulfonic acid.

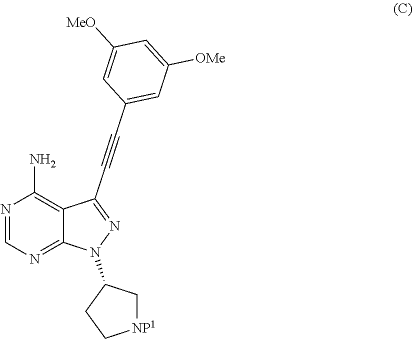

In the present invention, compound B or a salt thereof is obtained by deprotecting P¹ (P¹ representing a protecting group of an amino group) of a compound represented by formula (C). The compound represented by formula (C) can be obtained by the method disclosed in WO2013/108809.

Compound B in free form is easily soluble in water, highly water-soluble organic solvents, and highly fat-soluble organic solvents, whereas it is found that acid addition salts or base addition salts of compound B have low solubility in organic solvents and are easily isolated and purified.

Examples of the protecting group of an amino group represented by P¹ include protecting groups that can be deprotected under acidic conditions, such as a tert-butoxycarbonyl group (Boc group). The method for deprotection of P¹, which is a protecting group, can be suitably selected by those skilled in the art. When P¹ is a protecting group that can be deprotected under acidic conditions, such as a tert-butoxycarbonyl, the deprotection is preferably performed under acidic conditions. An acid such as hydrochloric acid, methanesulfonic acid, hydrogen iodide, or trifluoroacetic acid may be selected. Methanesulfonic acid is preferable in terms of reaction conditions, ease of use, burden on production equipment, and the like. The amount of acid used is, for example, preferably 1 to 100 moles per mole of the compound represented by formula (C).

For example, when P¹ is a protecting group that can be deprotected under acidic conditions, such as a tert-butoxycarbonyl, compound B can be obtained as an acid addition salt and can be converted to compound A or a pharmaceutically acceptable salt thereof.

In the present invention, compound A or a salt thereof is produced from compound B or a salt thereof by using an acryloylating reagent. As one embodiment of the acryloylating reagent of the present invention, a compound represented by the following formula (I-1-A) or formula (I-2-A) may be used.

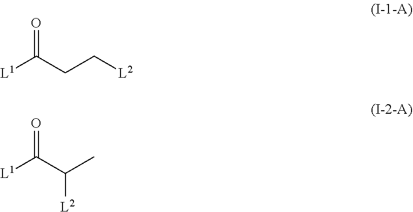

wherein L¹ and L² are the same or different, and each represents a leaving group.

In the compound represented by formula (I-1-A), leaving group L² is attached to the β-position of the carbonyl. In the compound represented by formula (I-2-A), leaving group L² is attached to the α-position of the carbonyl. In both cases, acryloyl group can be derived under basic conditions, and acrylamide in compound A can be constructed.

Examples of L¹, which is a leaving group, include halogen atoms and the like. L¹ is preferably a chlorine atom.

Examples of L², which is a leaving group, include halogen atoms, —OSO₂CₙFₙ₊₂ (n representing an integer of 1 to 4), mesylate (—OMs; Ms representing mesyl), tosylate (—OTs; Ts representing p-tosyl), nosylate (—ONs; Ns representing p-nosyl), —OSO₂Ph (Ph representing phenyl), phenoxy (—OPh), and the like. L² is preferably a halogen atom and more preferably a chlorine atom.

Examples of halogen atoms include a fluorine atom, a chlorine atom, a bromine atom, and an iodine atom.

Examples of the compound represented by formula (I-1-A) include 3-chloropropionyl chloride and 3-bromopropionyl chloride.

Examples of the compound represented by formula (I-2-A) include 2-chloropropionyl chloride and 2-bromopropionyl chloride.

As another embodiment of the acryloylating reagent of the present invention, a compound represented by the following formula (I-1-B), formula (I-1-C), formula (I-2-B), or formula (I-2-C) may be used. The compound represented by the following formula (I-1-B), formula (I-1-C), formula (I-2-B), or formula (I-2-C) is an acid anhydride.

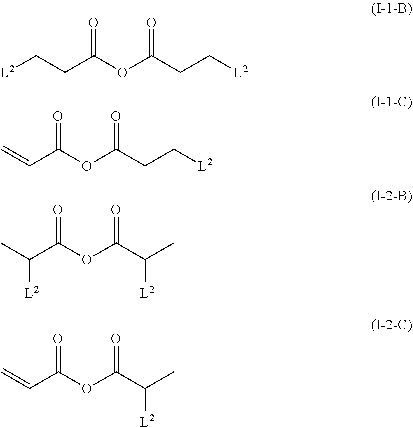

wherein each L² is the same or different, and each represents a leaving group.

Examples of L² include the leaving groups described above. L² is preferably a halogen atom and more preferably a chlorine atom.

Examples of the compound represented by formula (I-1-B) include 3-chloropropionic anhydride, 3-bromopropionic anhydride, 3-chloropropionic 3-bromopropionic anhydride, and the like. The compound represented by formula (I-1-B) is preferably 3-chloropropionic anhydride.

Examples of the compound represented by formula (I-1-C) include acrylic 3-chloropropionic anhydride, acrylic 3-bromopropionic anhydride, and the like. The compound represented by formula (I-1-C) is preferably acrylic 3-chloropropionic anhydride.

Examples of the compound represented by formula (I-2-B) include 2-chloropropionic anhydride, 2-bromopropionic anhydride, 2-chloropropionic 2-bromopropionic anhydride, and the like. The compound represented by formula (I-2-B) is preferably 2-chloropropionic anhydride.

Examples of the compound represented by formula (I-2-C) include acrylic 2-chloropropionic anhydride, acrylic 2-bromopropionic anhydride, and the like. The compound represented by formula (I-2-C) is preferably acrylic 2-chloropropionic anhydride.

In the present invention, the acryloylating reagent is preferably a compound represented by formula (I-1-A) or formula (I-2-A), more preferably a compound represented by formula (I-1-A), and even more preferably 3-chloropropionyl chloride.

In the present invention, the amount of the acryloylating reagent, which is a compound represented by formula (I-1-A), formula (I-1-B), formula (I-1-C), formula (I-2-A), formula (I-2-B), or formula (I-2-C), is 1.0 to 1.3 molar equivalents, more preferably 1.05 to 1.3 molar equivalents, and even more preferably 1.1 to 1.2 molar equivalents, per molar equivalent of compound B or a salt thereof. When a base containing a hydroxide ion is used, the acryloylating reagent may be used in an amount of not less than 1.0 molar equivalent, per molar equivalent of compound B or a salt thereof; the acryloylating reagent is preferably used in an amount of 1.0 to 3.0 molar equivalents, and more preferably 1.1 to 2.0 molar equivalents. In the method of the present invention, one —C(═O)—CH₂—CH₂-L² group is intended to be added per molecule of compound B or a salt thereof. Accordingly, in the present specification, for example, using 1.0 molar equivalent of the compound represented by formula (I-1-A), the compound represented by formula (I-1-C), or the compound represented by formula (I-2-C) per molar equivalent of compound B or a salt thereof means using 1.0 mole of the acryloylating reagent (acryloylating reagent having one —C(═O)—CH₂—CH₂-L² group per molecule) per mole of compound B or a salt thereof. Moreover, for example, using 1.0 molar equivalent of the compound represented by formula (I-1-B) or the compound represented by formula (I-2-B) per molar equivalent of compound B or a salt thereof means using 0.5 moles of the acryloylating reagent (acryloylating reagent having two —C(═O)—CH₂—CH₂-L² groups per molecule) per mole of compound B or a salt thereof. When these acryloylating reagents are used in combination, the above calculations are combined.

In the present specification, “equivalent” means molar equivalent unless it is obvious that it is meant otherwise.

In the present invention, the compound represented by formula (I-1-A), formula (I-1-B), formula (I-1-C), formula (I-2-A), formula (I-2-B), or formula (I-2-C) can be used as the acryloylating reagent. Thus, when the compound represented by formula (I-1-A) or formula (I-2-A) is used, the reaction proceeds in the following two steps, and compound A or a pharmaceutically acceptable salt thereof can be produced.

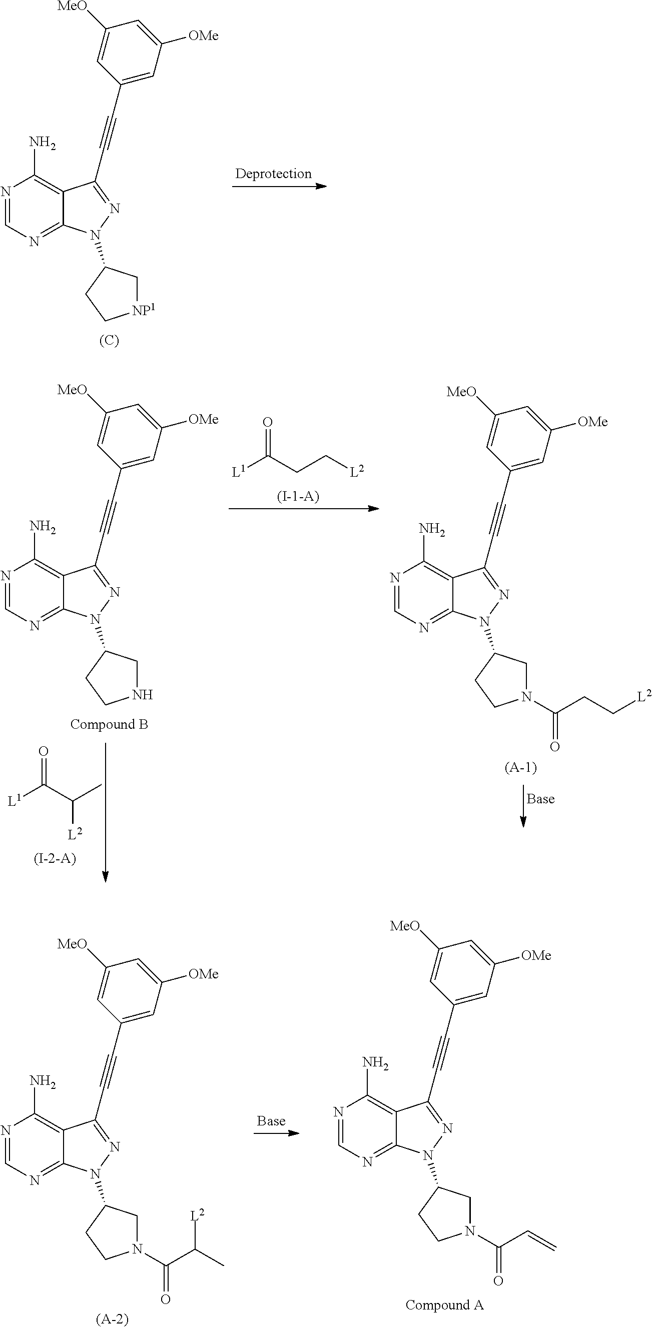

wherein L¹ and L² are the same as above.

When compound A or a pharmaceutically acceptable salt thereof is produced from compound B or a salt thereof, a compound represented by formula (A-1) or formula (A-2), or a salt thereof is obtained as an intermediate. In the present invention, L² can be eliminated from these intermediates to produce compound A without isolating the intermediates.

For example, when 3-chloropropionyl chloride is used as the compound represented by formula (I-1-A), the compound represented by formula (A-1) is (S)-1-(3-(4-amino-3-((3,5-dimethoxyphenyl)ethynyl)-1H-pyrazolo[3,4-d]pyrimidin-1-yl)pyrrolidin-1-yl)-3-chloropropan-1-one (which hereinafter may be referred to as “A-1-3CP compound”).

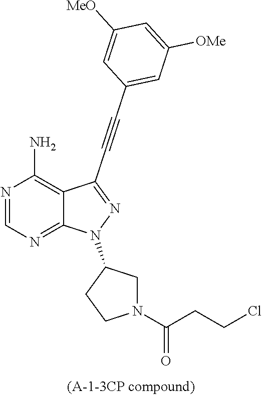 [SMILES: COc1cc(C#Cc2nn([C@H]3CCN(C(=O)CCCl)C3)c3ncnc(N)c23)cc(OC)c1]

When 2-chloropropionyl chloride is used as the compound represented by formula (I-2-A), the compound represented by formula (A-2) as an intermediate is 1-((S)-3-(4-amino-3-((3,5-dimethoxyphenyl)ethynyl)-1H-pyrazolo[3,4-d]pyrimidin-1-yl)pyrrolidin-1-yl)-2-chloropropan-1-one (which hereinafter may be referred to as “A-1-2CP compound”).

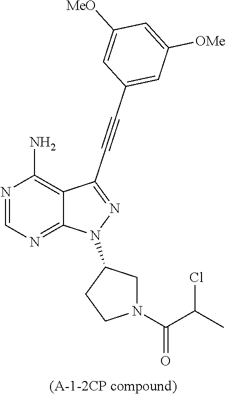 [SMILES: COc1cc(C#Cc2nn([C@H]3CCN(C(=O)C(C)Cl)C3)c3ncnc(N)c23)cc(OC)c1]

Compound B or a salt thereof, and the compound represented by formula (A-1) or formula (A-2), or a salt thereof can be used to confirm whether the reaction has proceeded when compound A is derived from compound B. Furthermore, since these compounds or salts thereof are possibly contained as impurities in compound A, they can also be used to determine the presence of impurities.

In the case of using compound A as a pharmaceutical product, the guideline on the amount of compound that can be contained as impurities in a drug substance and/or pharmaceutical preparation of the pharmaceutical product is indicated in the ICH-Q3 guideline of the International Council for Harmonisation of Technical Requirements for Pharmaceuticals for Human Use.

When acryloyl chloride is used in producing compound A or a pharmaceutically acceptable salt thereof from compound B or a salt thereof, (S)—N-(1-(1-acryloylpyrrolidin-3-yl)-3-((3,5-dimethoxyphenyl)ethynyl)-1H-pyrazolo[3,4-d]pyrimidin-4-yl)acrylamide (which hereinafter may be referred to as “diamide compound”) may be contained in compound A produced (drug substance of compound A).

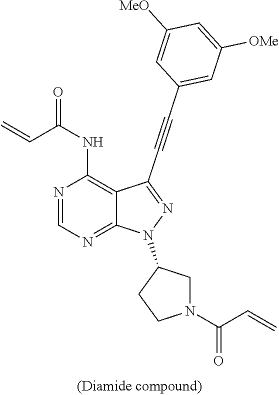 [SMILES: C=CC(=O)Nc1ncnc2c1c(C#Cc1cc(OC)cc(OC)c1)nn2[C@H]1CCN(C(=O)C=C)C1]

The present inventor tried a method in which acryloyl chloride is used in a smaller amount in order to remove the diamide compound or suppress the formation of the diamide compound, and found that with this method, compound B remained, resulting in a decrease in yield. The inventor also tried a method in which diamide is decomposed by adjusting the pH, and found that with this method, compound A could not be produced in large quantities efficiently because the number of steps was increased and that further, compound A was also decomposed, resulting in a decrease in yield. Furthermore, although crystallization conditions were examined, it was difficult to efficiently remove the diamide compound. In light of the above, it is believed that it is difficult to produce compound A in large quantities while maintaining the quality as a pharmaceutical product in the method for producing compound A by using acryloyl chloride.

Also when the compound represented by formula (A-1) or formula (A-2), or a salt thereof is derived from compound B or a salt thereof using the compound represented by formula (I-1-A), formula (I-1-B), formula (I-1-C), formula (I-2-A), formula (I-2-B), or formula (I-2-C) as the acryloylating reagent, the diamide compound may be obtained as a by-product via a compound represented by formula (A-1-diamide) or formula (A-2-diamide), as shown below.

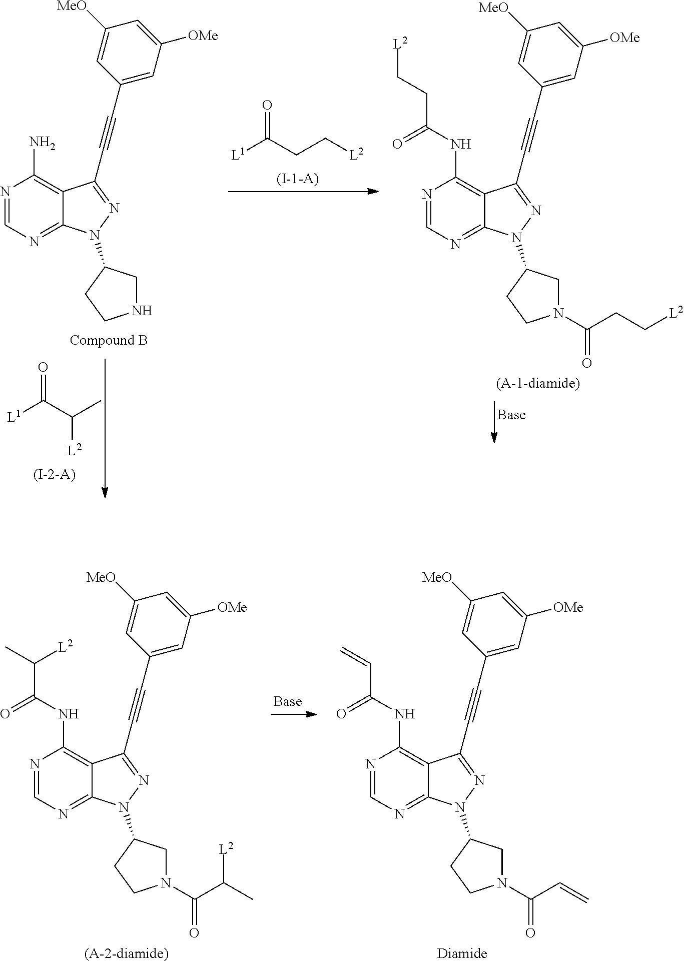

wherein L¹ and L² are the same as above.

The diamide or the compound represented by formula (A-1-diamide) or formula (A-2-diamide) can be used to determine the presence of impurities contained in compound A or a pharmaceutically acceptable salt thereof, the compound of formula (A-1) or a salt thereof, or the compound of formula (A-1) or a salt thereof.

For example, when L¹ and L² in the compound represented by formula (I-1-A), formula (I-1-B), formula (I-1-C), formula (I-2-A), formula (I-2-B), or formula (I-2-C) are chlorine, formula (A-1-diamide) is (S)-3-chloro-N-(1-(1-(3-chloropropanoyl)pyrrolidin-3-yl)-3-((3,5-dimethoxyphenyl)ethynyl)-1H-pyrazolo[3,4-d]pyrimidin-4-yl)propanamide (which hereinafter may be referred to as “3CP diamide”).

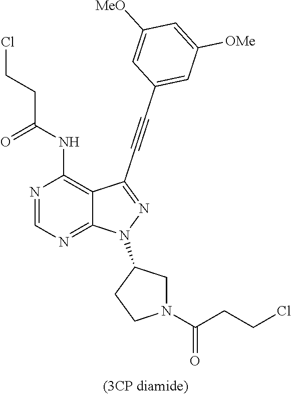 [SMILES: COc1cc(C#Cc2nn([C@H]3CCN(C(=O)CCCl)C3)c3ncnc(NC(=O)CCCl)c23)cc(OC)c1]

When L¹ and L² in the compound represented by formula (I-1-A), formula (I-1-B), formula (I-1-C), formula (I-2-A), formula (I-2-B), or formula (I-2-C) are chlorine, formula (A-2-diamide) is 2-chloro-N-(1-((3S)-1-(2-chloropropanoyl)pyrrolidin-3-yl)-3-((3,5-dimethoxyphenyl)ethynyl)-1H-pyrazolo[3,4-d]pyrimidin-4-yl)propanamide (which hereinafter may be referred to as “2CP diamide”).

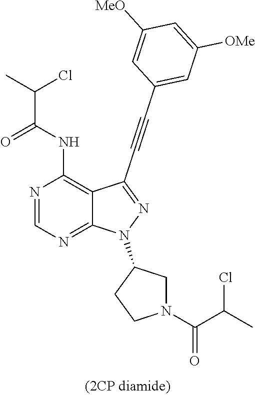 [SMILES: COc1cc(C#Cc2nn([C@H]3CCN(C(=O)C(C)Cl)C3)c3ncnc(NC(=O)C(C)Cl)c23)cc(OC)c1]

In the present invention, when the compound represented by formula (A-1) or formula (A-2), or a salt thereof is derived as an intermediate from compound B, it may be derived in the presence of a base (e.g., a base in an amount that is at least equivalent to that of compound B). Furthermore, when compound A is derived from these intermediates, it may be derived in the presence of a base (e.g., a base in an amount that is at least equivalent to that of the intermediate). When both steps are performed in the presence of a base, the bases in the steps may be the same or different from each other.

Examples of bases that can be used when the compound represented by formula (A-1) or formula (A-2), or a salt thereof is derived from compound B or a salt thereof include organic amine bases such as trimethylamine, triethylamine, diisopropylethylamine, N-methylmorpholine, diazabicycloundecene (DBU), diazabicyclononene (DBN), pyridine, and 4-dimethylaminopyridine (DMAP); inorganic bases such as lithium hydroxide, sodium hydroxide, magnesium hydroxide, potassium hydroxide, calcium hydroxide, cesium hydroxide, lithium acetate, sodium acetate, magnesium acetate, potassium acetate, calcium acetate, cesium acetate, lithium carbonate, sodium carbonate, magnesium carbonate, potassium carbonate, calcium carbonate, cesium carbonate, lithium hydrogencarbonate, sodium hydrogencarbonate, potassium hydrogencarbonate, cesium hydrogencarbonate, lithium phosphate, sodium phosphate, magnesium phosphate, potassium phosphate, calcium phosphate, and cesium phosphate; and the like. The organic amine bases or inorganic bases are preferable, bases containing a hydroxide ion are more preferable, bases containing an alkali metal ion (e.g., sodium ion or potassium ion) and a hydroxide ion are even more preferable, and sodium hydroxide or potassium hydroxide is still even more preferable. These bases may be used singly or in a combination of two or more.

Examples of bases that can be used when L² is eliminated from the compound represented by formula (A-1) or formula (A-2) to obtain compound A include the bases described above. The inorganic bases are preferable, bases containing a hydroxide ion are more preferable, bases containing an alkali metal ion (e.g., sodium ion or potassium ion) and a hydroxide ion are even more preferable, and sodium hydroxide or potassium hydroxide is still even more preferable.

The bases described above are roughly classified into monovalent bases, divalent bases, or trivalent bases. A monovalent base is a base that can accept one proton per molecule, and examples include triethylamine, diisopropylethylamine, sodium hydroxide, potassium hydroxide, sodium hydrogencarbonate, and the like. A divalent base is a base that can accept two protons per molecule, and examples include sodium carbonate and the like. A trivalent base is a base that can accept three protons per molecule, and examples include potassium phosphate and the like.

In the present invention, when a monovalent base is used in deriving the compound represented by formula (A-1) or formula (A-2), or a salt thereof from compound B or a salt thereof, the amount of the base is preferably 0.5 to 10 equivalents, more preferably 1 to 10 equivalents, even more preferably 1 to 5 equivalents, still even more preferably 1 to 3 equivalents, and particularly preferably 1 to 2 equivalents, after subtracting the equivalent amount neutralized with an acid addition salt of compound B, per equivalent of compound B or a salt thereof, i.e., relative to compound B in free form. When a monovalent base is used, the amount of the base used in eliminating L² from the compound represented by formula (A-1) or formula (A-2), or a salt thereof to obtain compound A or a pharmaceutically acceptable salt thereof is preferably 1 to 10 equivalents, and more preferably 1 to 5.0 equivalents, per equivalent of compound B or a salt thereof, i.e., relative to compound B in free form. Similarly, for divalent bases and trivalent bases, the optimal equivalent amounts can be calculated according to the above, taking the valence into account.

In the present invention, when a monovalent base is used in eliminating L² from the compound represented by formula (A-1) or formula (A-2), or a salt thereof to obtain compound A or a pharmaceutically acceptable salt thereof, the amount of the base may be 1 to 5 equivalents, after subtracting the equivalent amount neutralized with an acid addition salt of the compound represented by formula (A-1) or formula (A-2), per equivalent of the compound represented by formula (A-1) or formula (A-2), or a salt thereof, i.e., relative to the compound represented by formula (A-1) or formula (A-2) that is in free form. When the above process is performed without isolating the compound represented by formula (A-1) or formula (A-2), the amount of the base may be 1 to 10 equivalents relative to the theoretical yield of the compound represented by formula (A-1) or formula (A-2) that is in free form (i.e., the amount of compound B in free form). Similarly, for divalent bases and trivalent bases, the optimal equivalent amounts can be calculated according to the above, taking the valence into account.

In the present invention, the solvent used when the compound represented by formula (A-1) or formula (A-2), or a salt thereof is derived from compound B or a salt thereof is not particularly limited as long as it does not interfere with bonding of compound B to the acryloylating reagent. Examples of solvents include acetonitrile, water, N-methyl-2-pyrrolidone, tetrahydrofuran, acetone, N,N-dimethylformamide, N,N-dimethylacetamide, dimethyl sulfoxide, 1,4-dioxane, and mixtures thereof. Acetonitrile, water, or a mixture thereof is preferable. The volume of the solvent is not particularly limited, and the amount of the solvent is preferably 1 to 50 times by volume (v/w), more preferably 2 to 30 times by volume (v/w), and even more preferably 10 to 20 times by volume (v/w), per 1 weight of compound B or a salt thereof. When a mixture of solvents is used, the proportion of each solvent is not particularly limited. For example, when a mixture of acetonitrile and water is used, the proportion of each solvent is not particularly limited, and the amount of water is preferably 0.1 to 2 times by volume (v/v), more preferably 0.1 to 1 time by volume (v/v), and even more preferably 0.5 to 1 time by volume (v/v), per 1 volume of acetonitrile.

Examples of solvents that can be used when L² is eliminated from the compound represented by formula (A-1) or formula (A-2), or a salt thereof to obtain compound A or a pharmaceutically acceptable salt thereof include the same solvents as described above. Further, examples of solvents that can be used when this process is performed without isolating the compound represented by formula (A-1) or formula (A-2), or a salt thereof also include the same solvents as described above.

In the present invention, the temperature of the solvent used when the compound represented by formula (A-1) or formula (A-2), or a salt thereof is derived from compound B or a salt thereof is not particularly limited as long as it is between the melting point and the boiling point of the solvent and is within the range in which compound B can be stably present, and is preferably 0 to 50° C., and more preferably 25 to 35° C.

The temperature used when L² is eliminated from the compound represented by formula (A-1) or formula (A-2), or a salt thereof to obtain compound A or a pharmaceutically acceptable salt thereof is, for example, within the same range as described above.

In the present invention, a carboxylic acid represented by the following formula (I-1-D) or formula (I-2-D) may be used as a way to derive the compound represented by formula (A-1) or formula (A-2), or a salt thereof from compound B or a salt thereof.

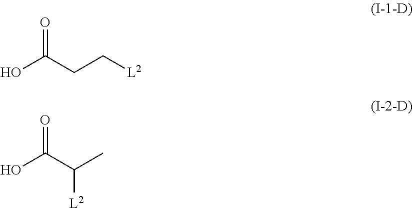

wherein L¹ and L² are the same as above.

In this case, the step in which the compound represented by formula (A-1) or formula (A-2), or a salt thereof can be derived from compound B or a salt thereof is as shown below.

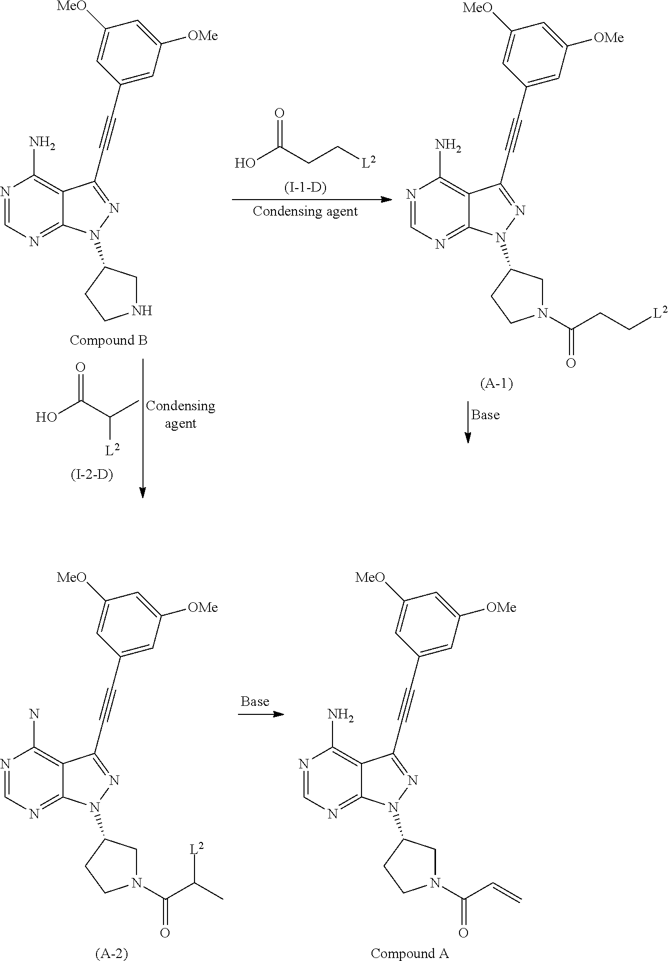

wherein L¹ and L² are the same as above.

Since this step is the condensation of compound B with the carboxylic acid of formula (I-1-D) or formula (I-2-D), a condensing agent can be used. Examples of condensing agents include dicyclohexylcarbodiimide (DCC), diisopropylcarbodiimide (DIC), 1-ethyl-3-(3-dimethylaminopropyl)carbodiimide hydrochloride (EDC, WSCI), benzotriazol-1-yloxy-tris-(dimethylamino)phosphonium hexafluorophosphate (BOP), benzotriazol-1-yloxy-tris-pyrrolidino-phosphonium hexafluorophosphate (PyBOP), (2-(7-aza-1H-benzotriazole-1-yl)-1,1,3,3,-tetramethyluronium hexafluorophosphate (HATU), 0-(1H-benzotriazol-1-yl)-N,N,N′,N′-tetramethyluronium hexafluorophosphate (HBTU), carbonyldiimidazole (CDI), 4-(4,6-dimethoxy-(1,3,5)triazin-2-yl)-4-methylmorpholinium chloride (DMTMM), and the like.

In this step, p-nitrophenol, pentafluorophenol, 2,4,5-trichlorophenol, 1-hydroxybenzotriazole (HOBt), 1-hydroxy-7-azabenzotriazole (HOAt), N-hydroxysuccinimide (HOSu), or the like may be added in order to convert the carboxylic acid to an activated ester.

Further, in this step, the bases listed above can be used as appropriate.

In the present invention, the reactions in deriving the compound represented by formula (A-1) or formula (A-2), or a salt thereof from compound B or a salt thereof and in eliminating L² from the compound represented by formula (A-1) or formula (A-2), or a salt thereof to obtain compound A or a pharmaceutically acceptable salt thereof may be confirmed by using, for example, chromatography, such as high-performance liquid chromatography (which hereinafter may be referred to as “HPLC”) and thin-layer chromatography (TLC). In the case of using HPLC, when the peak area of compound B is 1% or less of the total peak area, it can be determined that the step of deriving the compound represented by formula (A-1) or formula (A-2), or a salt thereof from compound B or a salt thereof is complete. Further, when the peak area of the compound represented by formula (A-1) or formula (A-2) is 1% or less of the total peak area, it can be determined that the step of eliminating L² from the compound represented by formula (A-1) or formula (A-2), or a salt thereof to obtain compound A is complete. The measurement conditions of HPLC are not particularly limited as long as compound A, compound B, and the compound represented by formula (A-1) or formula (A-2) can be detected.

In addition, in the case of using HPLC to confirm the reaction in deriving the compound represented by formula (A-1) or formula (A-2), or a salt thereof from compound B or a salt thereof, when the peak area of impurities and 3CP diamide represented by formula (A-1-diamide) or impurities and 2CP diamide represented by formula (A-2-diamide) is 2% or less of the total peak area, the diamide compound is less likely to be contained in compound A or a pharmaceutically acceptable salt thereof.

In the present invention, when the compound represented by formula (A-1) or formula (A-2), or a salt thereof is isolated, these compounds may be purified by a method such as recrystallization or may be used in the next step without purification. In addition, after the completion of the process in which L² is eliminated from the compound represented by formula (A-1) or formula (A-2), or a salt thereof to obtain compound A or a pharmaceutically acceptable salt thereof, compound A or a pharmaceutically acceptable salt thereof may be purified. As a purification method, from the viewpoint that the present invention is used for production in large quantities, it is preferable to use crystallization without performing purification by column chromatography.

In the present invention, when compound A or a pharmaceutically acceptable salt thereof dissolved in the reaction solvent is crystallized, for example, a solvent in which compound A or a pharmaceutically acceptable salt thereof has low solubility may be added. Examples of the solvent added include water and the like. The amount of the solvent added is not particularly limited as long as compound A or a pharmaceutically acceptable salt thereof is precipitated, and is preferably 0.5 to 5 times by volume (v/v), more preferably 1 to 3 times by volume (v/v), and even more preferably 1.5 to 2 times by volume (v/v), relative to the volume of the reaction solvent. When compound A or a pharmaceutically acceptable salt thereof is produced from compound B or a salt thereof without isolating the compound represented by formula (A-1) or formula (A-2), or a salt thereof, the amount of the solvent added is 5 to 50 times by volume (v/w), preferably 10 to 40 times by volume (v/w), and more preferably 15 to 30 times by volume (v/w), relative to the weight of compound B or a salt thereof.

In the present invention, the temperature at which crystallization is performed is not particularly limited as long as compound A or a pharmaceutically acceptable salt thereof is precipitated after the addition of the above solvent, and is preferably 0 to 40° C., and more preferably 20 to 30° C.

Furthermore, in the present invention, the time required for crystallization is, for example, 1 hour or more, and preferably 2 to 72 hours.

In the present invention, compound A or a pharmaceutically acceptable salt thereof may be isolated as a solid by crystallization and filtration. Since compound A or a pharmaceutically acceptable salt thereof is used as a pharmaceutical product, the time required for filtration is preferably short in order to efficiently produce it in large quantities. Since whether filterability is good or bad cannot be determined according to the absolute values of the filtration time, the filtration rate, and the like, it is determined relatively by comparing process conditions. Thus, the filtration areas, filter paper used for filtration, and pressures during suction are made uniform for comparison. No filter paper clogging caused by the precipitation of particles due to their large size, a small amount of solvent filtered, and the like are factors from which it can be determined that the filterability is excellent. Accordingly, in the present invention, improvement in filterability means shortening the filtration time (increasing the filtration rate) in filtration of compound A or a pharmaceutically acceptable salt thereof by using compound B or a pharmaceutically acceptable salt thereof and the compound represented by formula (I-1-A) in the equivalent ratio described above (and, if the type, amount, etc. of base is changed, then using the base as described above), without changing other process conditions (e.g., filtration area, filter paper used for filtration, and pressure during suction).

In the present invention, the filterability in filtration of compound A or a pharmaceutically acceptable salt thereof is better when the compound of formula (I-1-A) or formula (I-2-A) is used in producing compound A or a pharmaceutically acceptable salt thereof from compound B or a salt thereof without isolating the compound represented by formula (A-1) or formula (A-2), or a salt thereof, than when acryloyl chloride is used. This would not have been predicted when producing compound A or a salt thereof. In the present invention, the compound represented by formula (I-1-A) or formula (I-2-A) that can be used in terms of filterability is not particularly limited as long as the filterability is improved compared with the case of using acryloyl chloride, and is preferably the compound represented by formula (I-1-A), and more preferably 3-chloropropionyl chloride.

The thus obtained compound represented by formula (A-1) or formula (A-2), or a salt thereof, and compound A or a pharmaceutically acceptable salt thereof can be analyzed by various quantitative analyses and qualitative analyses.

In the present invention, as one embodiment, a method for producing the compound represented by formula (A-1) or a salt thereof, the method comprising reacting 1 equivalent of compound B or a salt thereof with 1.0 to 1.3 equivalents of the compound represented by formula (I-1-A), may be used.

Preferably, the method is for producing the compound represented by formula (A-1) or a salt thereof, comprising reacting 1 equivalent of compound B or a salt thereof with 1.0 to 1.3 equivalents of the compound represented by formula (I-1-A) wherein L¹ and L² are the same or different, and each is selected from the group consisting of halogen atoms.

More preferably, the method is for producing the compound represented by formula (A-1) or a salt thereof, comprising reacting 1 equivalent of compound B or a salt thereof with 1.0 to 1.3 equivalents of 3-chloropropionyl chloride.

More preferably, the method is for producing the compound represented by formula (A-1) or a salt thereof, comprising reacting 1 equivalent of compound B or a salt thereof with 1.0 to 1.3 equivalents of 3-chloropropionyl chloride in the presence of at least one base selected from the group consisting of organic amine bases and inorganic bases.

More preferably, the method is for producing the compound represented by formula (A-1) or a salt thereof, comprising reacting 1 equivalent of compound B or a salt thereof with 1.0 to 1.3 equivalents of 3-chloropropionyl chloride in the presence of at least one base selected from the group consisting of organic amine bases and bases containing a hydroxide ion.

More preferably, the method is for producing the compound represented by formula (A-1) or a salt thereof, comprising reacting 1 equivalent of compound B or a salt thereof with 1.0 to 1.3 equivalents of 3-chloropropionyl chloride in the presence of at least one base selected from the group consisting of organic amine bases and bases containing a hydroxide ion, the amount of the base after subtracting the equivalent amount neutralized with an acid addition salt of compound B or a salt thereof being 1.0 to 10 equivalents.

More preferably, the method is for producing the compound represented by formula (A-1) or a salt thereof, comprising reacting 1 equivalent of compound B or a salt thereof with 1.0 to 1.3 equivalents of 3-chloropropionyl chloride in the presence of at least one base selected from the group consisting of organic amine bases and bases containing an alkali metal ion and a hydroxide ion, the amount of the base after subtracting the equivalent amount neutralized with an acid addition salt of compound B or a salt thereof being 0.5 to 10 equivalents.

More preferably, the method is for producing the compound represented by formula (A-1) or a salt thereof, comprising reacting 1 equivalent of compound B or a salt thereof with 1.0 to 1.3 equivalents of 3-chloropropionyl chloride in the presence of at least one base selected from the group consisting of diisopropylethylamine, sodium hydroxide, and potassium hydroxide, the amount of the base after subtracting the equivalent amount neutralized with an acid addition salt of compound B or a salt thereof being 0.5 to 10 equivalents.

More preferably, the method is for producing the compound represented by formula (A-1) or a salt thereof, comprising reacting 1 equivalent of compound B or a salt thereof with 1.05 to 1.2 equivalents of 3-chloropropionyl chloride in the presence of at least one base selected from the group consisting of diisopropylethylamine, sodium hydroxide, and potassium hydroxide, the amount of the base after subtracting the equivalent amount neutralized with an acid addition salt of compound B or a salt thereof being 0.5 to 10 equivalents.

In the present invention, as another embodiment, a method for producing the compound represented by formula (A-1) or a salt thereof, the method comprising reacting compound B or a salt thereof with the compound represented by formula (I-1-A) in the presence of a base containing a hydroxide ion, may be used.

Preferably, the method is for producing the compound represented by formula (A-1) or a salt thereof, comprising reacting compound B or a salt thereof with the compound represented by formula (I-1-A) wherein L¹ and L² are the same or different, and each is a halogen atom, in the presence of a base containing a hydroxide ion.

More preferably, the method is for producing the compound represented by formula (A-1) or a salt thereof, comprising reacting compound B or a salt thereof with 3-chloropropionyl chloride in the presence of a base containing a hydroxide ion.

More preferably, the method is for producing the compound represented by formula (A-1) or a salt thereof, comprising reacting 1 equivalent of compound B or a salt thereof with 3-chloropropionyl chloride in the presence of a base containing a hydroxide ion, the amount of the base after subtracting the equivalent amount neutralized with an acid addition salt of compound B or a salt thereof being 0.5 to 10 equivalents.

More preferably, the method is for producing the compound represented by formula (A-1) or a salt thereof, comprising reacting 1 equivalent of compound B or a salt thereof with 3-chloropropionyl chloride in the presence of a base containing an alkali metal ion and a hydroxide ion, the amount of the base after subtracting the equivalent amount neutralized with an acid addition salt of compound B or a salt thereof being 0.5 to 10 equivalents.

More preferably, the method is for producing the compound represented by formula (A-1) or a salt thereof, comprising reacting 1 equivalent of compound B or a salt thereof with 3-chloropropionyl chloride in the presence of at least one base selected from the group consisting of sodium hydroxide and potassium hydroxide, the amount of the base after subtracting the equivalent amount neutralized with an acid addition salt of compound B or a salt thereof being 0.5 to 10 equivalents.

More preferably, the method is for producing the compound represented by formula (A-1) or a salt thereof, comprising reacting 1 equivalent of compound B or a salt thereof with 3-chloropropionyl chloride in the presence of at least one base selected from the group consisting of sodium hydroxide and potassium hydroxide, the amount of the base after subtracting the equivalent amount neutralized with an acid addition salt of compound B or a salt thereof being 1.0 to 5.0 equivalents.

### EXAMPLES

The present invention is described in more detail below with reference to Examples. However, the present invention is not limited to the Examples. Although the present invention is sufficiently described by the Examples, a person skilled in the art will understand that various changes and modifications are available. Thus, such changes and modifications are included in the present invention unless they depart from the spirit and principal concept of the present invention.

The reagents used in the Examples are commercially available products unless indicated otherwise.

The yield in the Examples, Production Examples, and Comparative Examples was calculated as below:

the yield (%)=((the amount of a desired product obtained)/(the theoretical amount of the desired product obtained))×100.

The LCMS spectra were measured by using an ACQUITY SQD (quadrupole, produced by Waters Corporation) under the following conditions.

Column: ACQUITY UPLC® BEH C18, produced by Waters Corporation, 2.1×50 mm, 1.7 μm
MS Detection: ESI positive

### UV Detection: 254 and 280 nm

Column Flow Rate: 0.5 mL/minute
Mobile Phase: Water/acetonitrile (0.1% formic acid)

### Injection Volume: 1 μL

The conditions for performing HPLC in each step are as described below.

### Condition 1

### Column: InertSustain C18 (4.6 mm I.D.×150 mm, 3 μm)

Detector: Ultraviolet absorption photometer (measurement
wavelength: 287 nm)
Column Temperature: Constant temperature around 40° C.
Flow Rate: 1.0 mL/minute

### Injection Volume: 10 μL

Analysis Time: 25 minutes (Time span of measurement: 15 minutes)

### Mobile Phase:

Solution A: 10 mmol/L aqueous phosphoric acid solution

Solution B: Acetonitrile

### Gradient Program

TABLE 1

| Time (Min) | Mobile Phase A (%) | Mobile Phase B (%) |
|---|---|---|
| 0.00 | 95 | 5 |
| 8.00 | 20 | 80 |
| 15.00 | 20 | 80 |
| 15.01 | 95 | 5 |
| 25.00 | 95 | 5 |

### Condition 2

### Column: InertSustain C18 (4.6 mm I.D.×150 mm, 3 μm)

Detector: Ultraviolet absorption photometer (measurement
wavelength: 287 nm)
Column Temperature: Constant temperature around 40° C.
Flow Rate: 1.0 mL/minute

### Injection Volume: 10 μL

Analysis Time: 30 minutes (Time span of measurement: 15 minutes)

### Mobile Phase:

Solution A: 10 mmol/L aqueous phosphoric acid solution

Solution B: Acetonitrile

### Gradient Program

TABLE 2

| Time (Min) | Mobile Phase A (%) | Mobile Phase B (%) |
|---|---|---|
| 0.00 | 95 | 5 |
| 0.50 | 68 | 32 |
| 3.50 | 8 | 32 |
| 8.00 | 40 | 60 |
| 9.00 | 20 | 80 |
| 20.00 | 20 | 80 |
| 20.01 | 95 | 5 |
| 30.00 | 95 | 5 |

### Condition 3

### Column: InertSustain C18 (4.6 mm I.D.×150 mm, 3 μm)

Detector: Ultraviolet absorption photometer (measurement
wavelength: 220 nm)
Column Temperature: Constant temperature around 40° C.
Flow Rate: 1.0 mL/minute

### Injection Volume: 10 μL

Analysis Time: 78 minutes (Time span of measurement: 58 minutes)

### Mobile Phase:

Solution A: 10 mmol/L aqueous phosphoric acid solution

Solution B: Acetonitrile

### Gradient Program

TABLE 3

| Time (Min) | Mobile Phase A (%) | Mobile Phase B (%) |
|---|---|---|
| 0.00 | 95 | 5 |
| 5.00 | 70 | 30 |
| 40.00 | 70 | 30 |
| 53.00 | 20 | 80 |
| 68.00 | 20 | 80 |
| 68.01 | 95 | 5 |
| 78.00 | 95 | 5 |

### Production Example 1: Synthesis of (S)-3-((3,5-dimethoxyphenyl)ethynyl)-1-(pyrrolidin-3-yl)-1H-pyrazolo[3,4-d]pyrimidine-4-amine.2 methanesulfonate

Toluene (2165 g), tert-butyl(S)-3-(4-amino-3-iodo-1H-pyrazolo[3,4-d]pyrimidin-1-yl)pyrrolidine-1-carboxylate (500 g), 1-ethynyl-3,5-dimethoxybenzene (207.5 g, PTL 4), and triethylamine (176.35 g) were dissolved, and the inside of the reaction system was purged with nitrogen. Copper iodide (885 mg), bis(triphenylphosphine palladium dichloride (3.264 g), and triphenylphosphine (1.2195 g) were added to the dissolved mixture, and the mixture was stirred at an internal temperature of 75° C. for 18 hours in a nitrogen atmosphere. Thereafter, the reaction solution was partially taken out and measured by HPLC (condition 1). The peak area of tert-butyl(S)-3-(4-amino-3-iodo-1H-pyrazolo[3,4-d]pyrimidin-1-yl)pyrrolidine-1-carboxylate was confirmed to be 1.0% or less of the total peak area.

The internal temperature was cooled to 50° C., and ethyl acetate (2255 g), Scavenger SH Silica (250 g), and refined Shirasagi activated carbon (50 g) were added to the mixture, followed by stirring for 21 hours. The Scavenger SH Silica and refined SHIRASAGI® (SEISEI) activated carbon were removed from the mixture by suction filtration with a Nutsche filter. The residue was washed with 4 L of ethyl acetate and then mixed with the filtrate. The solvent was distilled off from the resulting filtrate under reduced pressure. 2.5 L of acetonitrile was added thereto when 6 L was distilled. The solvent was further distilled under reduced pressure. After distillation of 2.4 L, 2.5 L of acetonitrile was added. The solvent was further distilled under reduced pressure. After distillation of 2.6 L, acetonitrile was added so that the total amount was 5-fold by volume (v/w) relative to tert-butyl(S)-3-(4-amino-3-iodo-1H-pyrazolo[3,4-d]pyrimidin-1-yl)pyrrolidine-1-carboxylate.

Purified water (500 g) and methanesulfonic acid (234.5 g) were added to the obtained mixture, and the mixture was stirred at an internal temperature of 60° C. or more for 2 hours. 8 L of acetonitrile was added to the mixture over a period of 5 minutes to crystalize (S)-3-((3,5-dimethoxyphenyl)ethynyl)-1-(pyrrolidin-3-yl)-1H-pyrazolo[3,4-d]pyrimidine-4-amine.2 methanesulfonate. The mixture was cooled from an internal temperature of 52° C. to 25° C. over a period of 3 hours, and then stirred for 11 hours. Wet crystals of (S)-3-((3,5-dimethoxyphenyl)ethynyl)-1-(pyrrolidin-3-yl)-1H-pyrazolo[3,4-d]pyrimidine-4-amine.2 methanesulfonate were obtained from the mixture by suction filtration using a Nutsche filter. The wet crystals were washed with 2 L of acetonitrile and dried under reduced pressure at 60° C., thereby obtaining the title compound (571.56 g, yield 88.4%) as light yellow white crystals.

1H-NMR (400 MHz, D₂O) δ8.358 (s, 1H), 6.741 (d, 2.4 Hz, 2H), 6.539 (t, 2.2 Hz, 1H), 5.715-5.662 (m, 1H), 3.955-3.860 (m, 2H), 3.828 (s, 6H), 3.799-3.645 (m, 2H), 2.838 (s, 6H), 2.766-2.666 (m, 1H), 2.536-2.471 (m, 1H)

### Production Example 2: Synthesis of (S)—N-(1-(1-acryloylpyrrolidin-3-yl)-3-((3,5-dimethoxyphenyl)ethynyl)-1H-pyrazolo[3,4-d]pyrimidin-4-yl)acrylamide

Compound A (348 mg, PTL 1) and DMF (6 mL) were placed in a reaction vessel, and 60% mineral oil-containing sodium hydride (49 mg) was added thereto. Thereafter, 3-chloropropionyl chloride (120 μL) was added dropwise, and the mixture was stirred at room temperature for 30 minutes. The reaction solution was added to a saturated aqueous solution of sodium hydrogen carbonate, and extraction was performed with ethyl acetate, followed by distilling off the solvent of the organic layer. The residue was purified by column chromatography (Biotage SNAP Ultra HP-Sphere, chloroform/methanol), thereby obtaining the title compound (49 mg).

1H-NMR (400 MHz, CDCl₃) δ8.84 (1H, s), 8.71 (1H, d, J=1.2 Hz, NH), 7.37 (1H, ddd, J=17.0, 10.2, 5.1 Hz), 6.86 (2H, dd, J=2.2, 0.7 Hz), 6.74-6.48 (1H, m), 6.60-6.42 (1H, m), 6.56 (1H, dd, J=4.4, 2.2 Hz), 6.46-6.39 (1H, m), 5.96 (1H, ddd, J=10.4, 4.2, 1.2 Hz), 5.77-5.69 (1H, m), 5.68-5.56 (1H, m), 4.20-4.01 (2H, m), 4.15-3.70 (2H, m), 3.84 (6H, s), 2.75-2.43 (2H, m); m/z 473[M+H]+

### Production Example 3: Synthesis of (S)-1-(3-(4-amino-3-((3,5-dimethoxy)ethynyl)-1H-pyrazolo[3,4-d]pyrimidin-1-yl)pyrrolidin-1-yl)-3-chloropropan-1-one

(S)-3-((3,5-dimethoxyphenyl)ethynyl)-1-(pyrrolidin-3-yl)-1H-pyrazolo[3,4-d]pyrimidine-4-amine.2 methanesulfonate (1.0 g) and dimethyl sulfoxide (25 mL) were placed in a reaction vessel, and then a 1M aqueous sodium hydroxide solution (3.6 mL) was added thereto. 3-Chloropropanecarboxylic acid (297 mg) and DMT-MM hydrate (694 mg) were added thereto, followed by stirring at room temperature for 2 hours. 3-Chloropropanecarboxylic acid (67 mg) and DMT-MM hydrate (272 mg) were further added, and the mixture was stirred at room temperature for 1 hour. The reaction solution was added to an aqueous solution of sodium hydrogen carbonate, and extraction was performed with ethyl acetate, followed by distilling off the solvent of the organic layer. The residue was purified by column chromatography (Biotage SNAP Ultra HP-Sphere, chloroform/methanol), thereby obtaining the title compound (713 mg).

1H-NMR (400 MHz, DMSO-d₆) δ8.26 (1H, s), 7.98 (1H, brs), 6.90 (2H, m), 6.78 (1H, brs), 6.60 (1H, m), 5.55-5.38 (1H, m), 4.05-3.76 (2H, m), 3.85-3.74 (2H, m), 3.80-3.49 (2H, m), 3.77 (6H, s), 2.79 (2H, dt, J=25.8, 6.8 Hz), 2.49-2.28 (2H, m); m/z 455, 457[M+H]+

### Example 1: Synthesis of (S)-1-(3-(4-amino-3-((3,5-dimethoxyphenyl)ethynyl)-1H-pyrazolo[3,4-d]pyrimidin-1-yl) pyrrolidin-1-yl)-2-propen-1-one

(S)-3-((3,5-dimethoxyphenyl)ethynyl)-1-(pyrrolidin-3-yl)-1H-pyrazolo[3,4-d]pyrimidine-4-amine.2 methanesulfonate (10 g), water (56 mL), and acetonitrile (50 mL) were placed in a reaction vessel, and a 5N aqueous sodium hydroxide solution (14 mL) was added thereto. A solution prepared by diluting 3-chloropropionyl chloride (2.51 g) with acetonitrile (20 mL) was added, and the mixture was stirred at a temperature of 20 to 30° C. for 2 hours. The reaction solution was partially taken out and measured by HPLC (condition 2). The peak area of compound B was confirmed to be less than 0.1% of the total peak area. At this stage, the diamide compound and the 3CP diamide compound were not detected in HPLC. Thereafter, a 5N aqueous sodium hydroxide solution (4 mL) was further added, and the mixture was stirred at a temperature of 20 to 30° C. for 2 hours. Thereafter, a 5N aqueous sodium hydroxide solution (2 mL) was further added, and the mixture was stirred at a temperature of 20 to 30° C. for 2 hours. The reaction solution was partially taken out and measured by HPLC (condition 2). The peak area of the A-1-3CP compound was confirmed to be less than 0.1% of the total peak area. After completion of the reaction, water (150 mL) was added, and the insoluble matter was collected by filtration, followed by washing with water (50 mL) and acetonitrile (50 mL). The collected matter was dried under reduced pressure at 60° C., thereby obtaining the title compound (5.60 g, yield 74.5%).

Analysis of the obtained title compound by HPLC detected no diamide compound.

### Example 2: Synthesis of (S)-1-(3-(4-amino-3-((3,5-dimethoxyphenyl)ethynyl)-1H-pyrazolo[3,4-d]pyrimidin-1-yl) pyrrolidin-1-yl)-2-propen-1-one

(S)-3-((3,5-dimethoxyphenyl)ethynyl)-1-(pyrrolidin-3-yl)-1H-pyrazolo[3,4-d]pyrimidine-4-amine.2 methanesulfonate (25.0 g), water (69 mL), and acetonitrile (158 mL) were placed in a reaction vessel, and a 5N aqueous sodium hydroxide solution (35 mL) was added thereto. A solution prepared by diluting 3-chloropropionyl chloride (6.27 g) with acetonitrile (50 mL) was added over a period of 10 minutes. After completion of the dropwise addition, the mixture was stirred at 30° C. for 30 minutes. The reaction solution was partially taken out and measured by HPLC (condition 3). The peak area of compound B was confirmed to be less than 0.1% of the total peak area. At this stage, the diamide compound and the 3CP diamide compound were not detected in HPLC. Thereafter, a 5N aqueous sodium hydroxide solution (25 mL) was further added, and the mixture was stirred at 30° C. for 4 hours. The reaction solution was partially taken out and measured by HPLC (condition 3). The peak area of the A-1-3CP compound was confirmed to be less than 0.1% of the total peak area. After completion of the reaction, water (550 mL) was added over a period of 2 hours. After completion of the dropwise addition, the internal temperature was adjusted to 25° C., and the mixture was stirred for 1.5 hours. The insoluble matter was collected by filtration and washed with water (125 mL), followed by drying the washed matter at 60° C. under reduced pressure, thereby obtaining the title compound (16.02 g, yield 85.3%).

Analysis of the obtained title compound by HPLC detected no diamide compound.

### Example 3: Synthesis of (S)-1-(3-(4-amino-3-((3,5-dimethoxyphenyl)ethynyl)-1H-pyrazolo[3,4-d]pyrimidin-1-yl) pyrrolidin-1-yl)-2-propen-1-one by using 3.1 equivalents of sodium hydroxide

(S)-3-((3,5-dimethoxyphenyl)ethynyl)-1-(pyrrolidin-3-yl)-1H-pyrazolo[3,4-d]pyrimidine-4-amine.2 methanesulfonate (3.0 g), water (9.15 mL), and acetonitrile (18.96 mL) were placed in a reaction vessel, and then a 5N aqueous sodium hydroxide solution (3.34 mL) was added thereto, followed by adjusting the internal temperature to 30° C. A solution prepared by diluting 3-chloropropionyl chloride (0.753 g) with acetonitrile (6 mL) was added over a period of 10 minutes. After completion of the dropwise addition, the mixture was stirred at 30° C. for 30 minutes. The reaction solution was partially taken out and measured by HPLC (condition 3). The peak area of compound B was confirmed to be less than 0.1% of the total peak area. At this stage, the diamide compound and the 3CP diamide compound were not detected in HPLC. Thereafter, a 5N aqueous sodium hydroxide solution (3.56 mL) was further added, and the mixture was stirred at 30° C. for 4 hours. The reaction solution was partially taken out and measured by HPLC (condition 3). The peak area of the A-1-3CP compound was confirmed to be less than 0.1% of the total peak area. After completion of the reaction, water (66 mL) was added over a period of 30 minutes, and the internal temperature was adjusted to 25° C., followed by stirring for 1 hour. The insoluble matter was collected by filtration and washed with water (15 mL), followed by drying the collected matter under reduced pressure at 60° C., thereby obtaining the title compound (1.874 g, yield 83.1%).

Analysis of the obtained title compound by HPLC detected no diamide compound.

### Example 4: Synthesis of (S)-1-(3-(4-amino-3-((3,5-dimethoxyphenyl)ethynyl)-1H-pyrazolo[3,4-d]pyrimidin-1-yl) pyrrolidin-1-yl)-2-propen-1-one by using N,N-diisopropylethylamine

(S)-3-((3,5-dimethoxyphenyl)ethynyl)-1-(pyrrolidin-3-yl)-1H-pyrazolo[3,4-d]pyrimidine-4-amine.2 methanesulfonate (3.0 g), water (12.48 mL), and acetonitrile (18.96 mL) were placed in a reaction vessel, and N,N-diisopropylethylamine (2.26 g) was added thereto, followed by adjusting the internal temperature to 30° C. A solution prepared by diluting 3-chloropropionyl chloride (0.753 g) with acetonitrile (6 mL) was added over a period of 10 minutes. After completion of the dropwise addition, the mixture was stirred at 30° C. for 30 minutes. The reaction solution was partially taken out and measured by HPLC (condition 3). The peak area of compound B was confirmed to be less than 1.0% of the total peak area. At this stage, the diamide compound and the 3CP diamide compound were not detected in HPLC. Thereafter, a 5N aqueous sodium hydroxide solution (6.47 mL) was further added, and the mixture was stirred at 30° C. for 4 hours and 30 minutes. After completion of the reaction, water (66 mL) was added over a period of 30 minutes, and the internal temperature was adjusted to 25° C., followed by stirring for 1 hour and 30 minutes. The insoluble matter was collected by filtration and washed with water (15 mL), followed by drying the washed matter under reduced pressure at 60° C., thereby obtaining the title compound (1.897 g, yield 84.2%).

Analysis of the obtained title compound by HPLC detected no diamide compound.

### Example 5: Synthesis of (S)-1-(3-(4-amino-3-((3,5-dimethoxyphenyl)ethynyl)-1H-pyrazolo[3,4-d]pyrimidin-1-yl) pyrrolidin-1-yl)-2-propen-1-one by using potassium hydroxide

(S)-3-((3,5-dimethoxyphenyl)ethynyl)-1-(pyrrolidin-3-yl)-1H-pyrazolo[3,4-d]pyrimidine-4-amine.2 methanesulfonate (500 mg), water (3.5 mL), and acetonitrile (3.5 mL) were placed in a reaction vessel, and then a 5N potassium hydroxide (equivalent to 280.7 mg) was added thereto. A solution prepared by diluting 3-chloropropionyl chloride (205.2 mg) with acetonitrile (1 mL) was added, and the mixture was stirred at a temperature of 20 to 30° C. for 30 minutes. The reaction solution was partially taken out and measured by HPLC (condition 2). The peak area of compound B was confirmed to be less than 1.0% of the total peak area. At this stage, the diamide compound and the 3CP diamide compound were not detected in HPLC.

This suggests that the method of Example 5 is excellent in terms of product quality and is suitable for mass production of a pharmaceutical product.

### Example 6: Synthesis of (S)-1-(3-(4-amino-3-((3,5-dimethoxyphenyl)ethynyl)-1H-pyrazolo[3,4-d]pyrimidin-1-yl) pyrrolidin-1-yl)-2-propen-1-one by using sodium hydroxide

(S)-3-((3,5-dimethoxyphenyl)ethynyl)-1-(pyrrolidin-3-yl)-1H-pyrazolo[3,4-d]pyrimidine-4-amine.2 methanesulfonate (500 mg), water (3.5 mL), and acetonitrile (3.5 mL) were placed in a reaction vessel, and a 5N sodium hydroxide (1.0 mL) was added thereto. A solution prepared by diluting 3-chloropropionyl chloride (205.2 mg) with acetonitrile (1 mL) was added, and the mixture was stirred at a temperature of 20 to 30° C. for 30 minutes. The reaction solution was partially taken out and measured by HPLC (condition 2). The peak area of compound B was confirmed to be less than 1.0% of the total peak area. At this stage, the peak area of the 3CP diamide compound was 0.31%.

This suggests that the method of Example 6 is excellent in terms of product quality and is suitable for mass production of a pharmaceutical product.

### Comparative Example 1: Synthesis of (S)-1-(3-(4-amino-3-((3,5-dimethoxyphenyl)ethynyl)-1H-pyrazolo[3,4-d]pyrimidin-1-yl) pyrrolidin-1-yl)-2-propen-1-one by using acryloyl chloride

(S)-3-((3,5-dimethoxyphenyl)ethynyl)-1-(pyrrolidin-3-yl)-1H-pyrazolo[3,4-d]pyrimidine-4-amine.2 methanesulfonate (5.0 g), N-methyl-2-pyrrolidone (50 mL), and potassium phosphate (3.43 g) were placed in a reaction vessel, and the internal temperature was adjusted to 10° C. or less. Acryloyl chloride (1.219 g) was added, and the mixture was stirred for 3 hours. The reaction solution was partially taken out and measured by HPLC (condition 2). The peak area of compound B was confirmed to be less than 1.0% of the total peak area, and the peak area of the diamide compound was confirmed to be 2% or more.

Thereafter, water (25 g) and phosphoric acid (880 mg) were added, and the mixture was stirred at a temperature of 20 to 30° C. for 3 hours. The reaction solution was partially taken out and measured by HPLC (condition 2). The peak area of the diamide compound was confirmed to be less than 2% of the total peak area.

Thereafter, the pH of the reaction solution was adjusted to 7 to 8 with a 20% aqueous potassium hydroxide solution. Ethanol (40 mL) was then added thereto, and the mixture was heated to an internal temperature of 50 to 60° C. to dissolve it. At an internal temperature of 50° C., crystal II of compound A (25.2 mg, PTL 8) was added, and the mixture was stirred for 3 hours. Thereafter, water (176.3 mL) was added dropwise over a period of 3 hours. The internal temperature was cooled from 50° C. to 25° C. over a period of 15 hours. At this stage, the entire solution containing a solution and insoluble matter was 375 mL. From this solution, the insoluble matter was collected by filtration and washed with water (30 mL), followed by drying the washed matter under reduced pressure at 60° C., thereby obtaining the title compound (2.936 g, yield 78.2%).

Analysis of the obtained title compound by HPLC detected no diamide compound.

However, when the insoluble matter was filtered off from the entire solution (375 mL), containing a solution and insoluble matter, by using a Nutsche filter with a filtration area of 12.56 cm², filtration took 1716 seconds, and the filtration rate was 0.2 mL/second. This filtration rate was about 1/20 of the filtration rate in the Examples, when compared in terms of the time period that took for filtration on the same scale as that of the Examples.

This suggests that the method of Comparative Example 1 took time for filtration of compound A, and indicates that the conditions may be insufficient as a method for mass production.

### Comparative Example 2

(S)-3-((3,5-dimethoxyphenyl)ethynyl)-1-(pyrrolidin-3-yl)-1H-pyrazolo[3,4-d]pyrimidine-4-amine.2 methanesulfonate (500 mg), water (2.5 mL), and acetonitrile (2.5 mL) were placed in a reaction vessel, and a 5N aqueous sodium hydroxide solution (1 mL) was added thereto. A solution prepared by diluting acryloyl chloride (162.9 mg) with acetonitrile (1 mL) was then added thereto, followed by stirring the mixture at ice cooling temperature for 30 minutes. The reaction solution was partially taken out and measured by HPLC (condition 2). The peak area of compound B was 1.94% of the total peak area, and the peak area of the diamide compound was 3.02% of the total peak area.

This suggests that the method of Comparative Example 2 may be insufficient as a method for producing a pharmaceutical product from the standpoint of product quality.

### Comparative Example 3

(S)-3-((3,5-dimethoxyphenyl)ethynyl)-1-(pyrrolidin-3-yl)-1H-pyrazolo[3,4-d]pyrimidine-4-amine.2 methanesulfonate (500 mg), water (3.5 mL), and acetonitrile (2.5 mL) were placed in a reaction vessel, and then potassium phosphate (343.8 mg) was added thereto. A solution prepared by diluting acryloyl chloride (97.8 mg) with acetonitrile (1 mL) was then added thereto, followed by stirring the mixture at ice cooling temperature for 60 minutes. The reaction solution was partially taken out and measured by HPLC (condition 2). The peak area of compound B was 17.79% of the total peak area, and the peak area of the diamide compound was 20.85% of the total peak area.

This suggests that the method of Comparative Example 3 may be insufficient as a method for mass production of compound A.

### Comparative Example 4

(S)-3-((3,5-dimethoxyphenyl)ethynyl)-1-(pyrrolidin-3-yl)-1H-pyrazolo[3,4-d]pyrimidine-4-amine.2 methanesulfonate (500 mg), water (3.5 mL), and acetonitrile (2.5 mL) were placed in a reaction vessel, and potassium phosphate (343.8 mg) was added thereto. A solution prepared by diluting acryloyl chloride (146.6 mg) with acetonitrile (1 mL) was then added thereto, followed by stirring the mixture at ice cooling temperature for 60 minutes. The reaction solution was partially taken out and measured by HPLC (condition 2). Although the peak area of compound B was confirmed to be less than 1.0% of the total peak area, the peak area of the diamide compound was 44.23% of the total peak area.

This suggests that the method of Comparative Example 4 may be insufficient as a method for mass production of compound A.

### Comparative Example 5

(S)-3-((3,5-dimethoxyphenyl)ethynyl)-1-(pyrrolidin-3-yl)-1H-pyrazolo[3,4-d]pyrimidine-4-amine.2 methanesulfonate (500 mg), water (3.5 mL), and acetonitrile (2.5 mL) were placed in a reaction vessel, and diisopropylethylamine (406.2 mg) was added thereto. A solution prepared by diluting acryloyl chloride (171 mg) with acetonitrile (1 mL) was then added thereto, followed by stirring the mixture at ice cooling temperature for 60 minutes. The reaction solution was partially taken out and measured by HPLC. Although the peak area of compound B was less than 0.1% of the total peak area, the peak area of the diamide compound was 2.04%. At this stage, despite the recrystallization in various ways, the content of the diamide compound in compound A could not be reduced to less than 0.1%.

This suggests that the method of Comparative Example 5 may be insufficient as a method for producing a pharmaceutical product from the standpoint of product quality.

### Comparative Example 6

(S)-3-((3,5-dimethoxyphenyl)ethynyl)-1-(pyrrolidin-3-yl)-1H-pyrazolo[3,4-d]pyrimidine-4-amine.2 methanesulfonate (500 mg), water (3.5 mL), and acetonitrile (3.5 mL) were placed in a reaction vessel, and potassium carbonate (620.6 mg) was added thereto. A solution prepared by diluting 3-chloropropionyl chloride (205.2 mg) with acetonitrile (1 mL) was then added, followed by stirring the mixture at a temperature of 20 to 30° C. for 30 minutes. The reaction solution was partially taken out and measured by HPLC (condition 2). Although the peak area of compound B was less than 1.0% of the total peak area, the peak area of the 3CP diamide compound was 3.41% of the total peak area.

This suggests that the method of Comparative Example 6 may be insufficient as a method for producing a pharmaceutical product from the standpoint of product quality.

### Comparative Example 7

(S)-3-((3,5-dimethoxyphenyl)ethynyl)-1-(pyrrolidin-3-yl)-1H-pyrazolo[3,4-d]pyrimidine-4-amine.2 methanesulfonate (500 mg), water (3.5 mL), and acetonitrile (3.5 mL) were placed in a reaction vessel, and potassium phosphate (343.1 mg) was added thereto. A solution prepared by diluting 3-chloropropionyl chloride (205.2 mg) with acetonitrile (1 mL) was then added, followed by stirring the mixture at a temperature of 20 to 30° C. for 30 minutes. The reaction solution was partially taken out and measured by HPLC (condition 2). Although the peak area of compound B was less than 1.0% of the total peak area, the peak area of the 3CP diamide compound was 6.58% of the total peak area.

This suggests that the method of Comparative Example 7 may be insufficient as a method for producing a pharmaceutical product from the standpoint of product quality.

### Comparative Example 8

(S)-3-((3,5-dimethoxyphenyl)ethynyl)-1-(pyrrolidin-3-yl)-1H-pyrazolo[3,4-d]pyrimidine-4-amine.2 methanesulfonate (500 mg), water (3.5 mL), and acetonitrile (3.5 mL) were placed in a reaction vessel, and triethylamine (318.0 mg) was added thereto. A solution prepared by diluting 3-chloropropionyl chloride (171.0 mg) with acetonitrile (1 mL) was then added, followed by stirring the mixture at a temperature of 20 to 30° C. for 30 minutes. The reaction solution was partially taken out and measured by HPLC (condition 2). Although the peak area of compound B was less than 1.0% of the total peak area, the peak area of the 3CP diamide compound was 1.68% of the total peak area, with many other kinds of related substances being detected.

This suggest that the method of Comparative Example 8 may be insufficient as a method for producing a pharmaceutical product from the standpoint of product quality.

### Comparative Example 9

(S)-3-((3,5-dimethoxyphenyl)ethynyl)-1-(pyrrolidin-3-yl)-1H-pyrazolo[3,4-d]pyrimidine-4-amine.2 methanesulfonate (500 mg), water (3.5 mL), and acetonitrile (3.5 mL) were placed in a reaction vessel, and pyridine (3551.6 mg) was added thereto. A solution prepared by diluting 3-chloropropionyl chloride (205.2 mg) with acetonitrile (1 mL) was then added, followed by stirring the mixture at a temperature of 20 to 30° C. for 30 minutes. The reaction solution was partially taken out and measured by HPLC (condition 2). The peak area of compound B was 43.72%, indicating that the reaction did not sufficiently proceed.

This suggests that the method of Comparative Example 9 may be insufficient as a method for mass production of compound A.

### Comparative Example 10

(S)-3-((3,5-dimethoxyphenyl)ethynyl)-1-(pyrrolidin-3-yl)-1H-pyrazolo[3,4-d]pyrimidine-4-amine.2 methanesulfonate (500 mg), water (3.5 mL), and acetonitrile (3.5 mL) were placed in a reaction vessel, and potassium tert-butoxide (503.8 mg) was added thereto. A solution prepared by diluting 3-chloropropionyl chloride (205.2 mg) with acetonitrile (1 mL) was then added, followed by stirring the mixture at a temperature of 20 to 30° C. for 30 minutes. The reaction solution was partially taken out and measured by HPLC (condition 2). Although the peak area of compound B was less than 1.0% of the total peak area, the peak area of the 3CP diamide compound was 4.66% of the total peak area.

This suggests that the method of Comparative Example 10 may be insufficient as a method for producing a pharmaceutical product from the standpoint of product quality.

The following table illustrates the results of production of the compound represented by formula (A-1) in which L² is a chlorine atom from 2 methanesulfonate of compound B.

TABLE 4

| | | | | | | | | Comparative | Comparative |
|---|---|---|---|---|---|---|---|---|---|
| | | Example 1 | Example 2 | Example 3 | Example 4 | Example 5 | Example 6 | Example 1 | Example 2 |
| Reaction | Reagent | 3-Chloropropionyl | 3-Chloropropionyl | 3-Chloropropionyl | 3-Chloropropionyl | 3-Chloropropionyl | 3-Chloropropionyl | Acryloyl Chloride | Acryloyl Chloride |
| Conditions | | Chloride | Chloride | Chloride | Chloride | Chloride | Chloride | | |
| | Reagent Equivalent | 1.10 | 1.10 | 1.10 | 1.10 | 1.80 | 1.80 | 1.50 | 2.00 |
| | Base | Sodium Hydroxide | Sodium Hydroxide | Sodium Hydroxide | Diisopropylethylamine | Potassium Hydroxide | Sodium Hydroxide | Potassium Phosphate | Sodium Hydroxide |
| | (Valence) | (Monovalent) | (Monovalent) | (Monovalent) | (Monovalent) | (Monovalent) | (Monovalent) | (Trivalent) | (Monovalent) |
| | Base Equivalent | 1.90 | 1.90 | 1.10 | 1.24 | 3.57 | 3.57 | 1.14 | 3.57 |
| | (After Acid Addition | | | | | | | | |
| | Salt Is Subtracted) | | | | | | | | |
| | Solvent | Acetonitrile/Water | Acetonitrile/Water | Acetonitrile/Water | Acetonitrile/Water | Acetonitrile/Water | Acetonitrile/Water | N- | Acetonitrile/Water |
| | | (1:1) | (2:1) | (2:1) | (10:1) | (1:1) | (1:1) | Methylpyrrolidone | (1:1) |
| | Temperature | 20-30° C. | 20-30° C. | 30° C. | 30° C. | 20-30° C. | 20-30° C. | 20-30° C. | Ice Cooling |
| | | | | | | | | | Temperature |
| HPLC | Compound B | Less than 1.0% | Less than 1.0% | Less than 1.0% | Less than 1.0% | Less than 1.0% | Less than 1.0% | N.A. | 1.94% |
| during | Diamide + 3CP | N.D. | N.D. | N.D. | N.D. | N.D. | 0.31% | N.A. | 3.02% |
| Reaction | Diamide | | | | | | | | |
| Yield of Compound A (%) | | 74.5 | 85.3 | 83.1 | 84.2 | N.A. | N.A. | 78.2 | N.A. |
| | | Comparative | Comparative | Comparative | Comparative | Comparative | Comparative | Comparative | Comparative |
| | | Example 3 | Example 4 | Example 5 | Example 6 | Example 7 | Example 8 | Example 9 | Example 10 |
| Reaction | Reagent | Acryloyl Chloride | Acryloyl Chloride | Acryloyl Chloride | 3-Chloropropionyl | 3-Chloropropionyl | 3-Chloropropionyl | 3-Chloropropionyl | 3-Chloropropionyl |
| Conditions | | | | | Chloride | Chloride | Chloride | Chloride | Chloride |
| | Reagent Equivalent | 1.20 | 1.80 | 1.40 | 1.80 | 1.80 | 1.50 | 1.80 | 1.80 |
| | Base | Potassium | Potassium | Diisopropylethylamine | Potassium Carbonate | Potassium Phosphate | Triethylamine | Pyridine | Potassium |
| | (Valence) | Phosphate | Phosphate | (Monovalent) | (Divalent) | (Trivalent) | (Monovalent) | (Monovalent) | Tert-Butoxide |
| | | (Trivalent) | (Trivalent) | | | | | | (Monovalent) |
| | Base Equivalent | 1.14 | 1.14 | 1.50 | 4.00 | 1.14 | 1.50 | 48.0 | 3.00 |
| | (After Acid Addition | | | | | | | | |
| | Salt Is Subtracted) | | | | | | | | |
| | Solvent | Acetonitrile/Water | Acetonitrile/Water | Acetonitrile/Water | Acetonitrile/Water | Acetonitrile/Water | Acetonitrile/Water | Acetonitrile/Water | Acetonitrile/Water |
| | | (1:1) | (1:1) | (1:1) | (1:1) | (1:1) | (1:1) | (1:1) | (1:1) |
| | Temperature | Ice Cooling | Ice Cooling | Ice Cooling | 30° C. | 20-30° C. | 20-30° C. | 20-30° C. | 20-30° C. |
| | | Temperature | Temperature | Temperature | | | | | |
| HPLC | Compound B | 17.79% | Less than 1.0% | Less than 1.0% | Less than 1.0% | Less than 1.0% | Less than 1.0% | 43.72% | Less than 1.0% |
| during | Diamide + 3CP | 20.85% | 44.23% | 2.04% | 3.41% | 6.58% | 1.68% | N.D. | 4.66% |
| Reaction | Diamide | | | | | | | | |

| | | | | | | | | |
|---|---|---|---|---|---|---|---|---|
| Yield of Compound A (%) | N.A. | N.A. | N.A. | N.A. | N.A. | N.A. | N.A. | N.A. |

The phrase “After Acid Addition Salt Is Subtracted” written for the base equivalent refers to a value determined by subtracting the number of equivalents of the base required for neutralization of methanesulfonic acid (acid addition part) of 2 methanesulfonate of compound B. The expression “N.D.” means “below the detection limit,” and the expression “N.A.” means that measurement was not performed.

The results indicate that the probability of containing diamide in compound A is low when 1.1 equivalents of 3-chloropropionyl chloride is used per equivalent of compound B. The results also indicate that when sodium hydroxide or potassium hydroxide is used as a base, and when 1.8 equivalents of 3-chloropropionyl chloride per equivalent of compound B is used, the probability of containing diamide in compound A is low.

1. A method for producing a compound represented by the following formula (A-1) or a salt thereof, the method comprising reacting 1 equivalent of (S)-3-((3,5-dimethoxyphenyl)ethynyl)-1-(pyrrolidin-3-yl)-1H-pyrazolo[3,4-d]pyrimidin-4-amine or a salt thereof with 1.0 to 1.3 equivalents of a compound represented by the following formula (I-1-A) 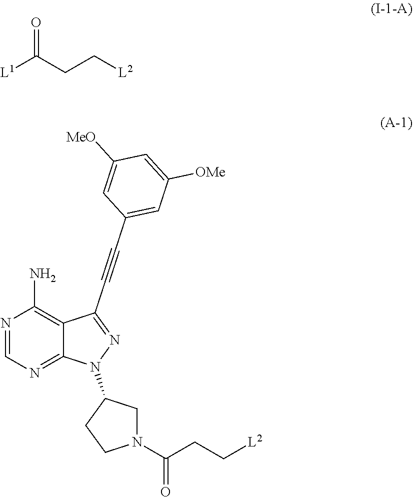
wherein L¹ and L² are the same or different, and each represents a leaving group.

2. The method according to claim 1, wherein L¹ and L² are the same or different, and each is a halogen atom.

3. The method according to claim 1, wherein the compound represented by formula (I-1-A) is 3-chloropropionyl chloride.

4. The method according to claim 1, wherein the reaction is performed in the presence of at least one base selected from the group consisting of organic amine bases and inorganic bases.

5. The method according to claim 4, wherein the base is a base containing a hydroxide ion.

6. The method according to claim 4, wherein the amount of the base after subtracting an equivalent amount neutralized with an acid addition salt of (S)-3-((3,5-dimethoxyphenyl)ethynyl)-1-(pyrrolidin-3-yl)-1H-pyrazolo[3,4-d]pyrimidin-4-amine or a salt thereof is 0.5 to 10 equivalents.

7. A method for producing (S)-1-(3-(4-amino-3-((3,5-dimethoxyphenyl)ethynyl)-1H-pyrazolo[3,4-d]pyrimidin-1-yl)pyrrolidin-1-yl)-2-propen-1-one or a pharmaceutically acceptable salt thereof, the method comprising:
producing a compound represented by formula (A-1) or a salt thereof by the method of claim 1; and
eliminating L² from the obtained compound represented by formula (A-1).

8. The method according to claim 7, wherein in the step of producing a compound represented by formula (A-1) or a salt thereof, a reaction is performed in the presence of at least one base selected from the group consisting of organic amine bases and inorganic bases.

9. The method according to claim 8, wherein the base is a base containing a hydroxide ion.

10. The method according to claim 8, wherein the amount of the base after subtracting an equivalent amount neutralized with an acid addition salt of (S)-3-((3,5-dimethoxyphenyl)ethynyl)-1-(pyrrolidin-3-yl)-1H-pyrazolo[3,4-d]pyrimidin-4-amine or a salt thereof is 0.5 to 10 equivalents per equivalent of (S)-3-((3,5-dimethoxyphenyl)ethynyl)-1-(pyrrolidin-3-yl)-1H-pyrazolo[3,4-d]pyrimidin-4-amine.

11. The method according to claim 1 for improving the yield of (S)-1-(3-(4-amino-3-((3,5-dimethoxyphenyl)ethynyl)-1H-pyrazolo[3,4-d]pyrimidin-1-yl)pyrrolidin-1-yl)-2-propen-1-one or a pharmaceutically acceptable salt thereof.

12. The method according to claim 11, wherein the yield of (S)-1-(3-(4-amino-3-((3,5-dimethoxyphenyl)ethynyl)-1H-pyrazolo[3,4-d]pyrimidin-1-yl)pyrrolidin-1-yl)-2-propen-1-one or a pharmaceutically acceptable salt thereof is improved by suppressing the formation of a diamide compound.

13. A method for improving filterability, the method comprising producing (S)-1-(3-(4-amino-3-((3,5-dimethoxyphenyl)ethynyl)-1H-pyrazolo[3,4-d]pyrimidin-1-yl)pyrrolidin-1-yl)-2-propen-1-one or a salt thereof by the method of claim 1.

14. A method for improving the yield of (S)-1-(3-(4-amino-3-((3,5-dimethoxyphenyl)ethynyl)-1H-pyrazolo[3,4-d]pyrimidin-1-yl)pyrrolidin-1-yl)-2-propen-1-one or a pharmaceutically acceptable salt thereof, the method comprising producing (S)-1-(3-(4-amino-3-((3,5-dimethoxyphenyl)ethynyl)-1H-pyrazolo[3,4-d]pyrimidin-1-yl)pyrrolidin-1-yl)-2-propen-1-one or a salt thereof by the method of claim 1.

15. A method for producing a compound represented by the following formula (A-1) or a salt thereof, the method comprising reacting (S)-3-((3,5-dimethoxyphenyl)ethynyl)-1-(pyrrolidin-3-yl)-1H-pyrazolo[3,4-d]pyrimidin-4-amine or a salt thereof with a compound represented by the following formula (I-1-A) in the presence of a base containing a hydroxide ion 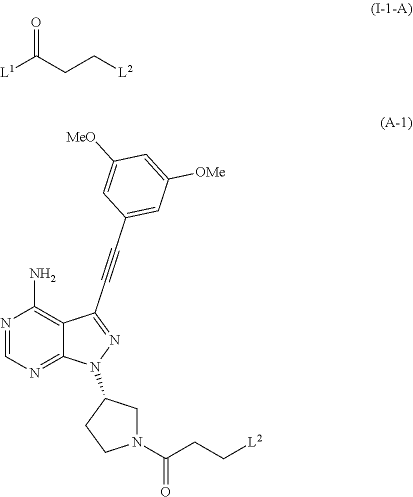
wherein L¹ and L² are the same or different, and each represents a leaving group.

16. The method according to claim 15, wherein L¹ and L² are the same or different, and each is a halogen atom.

17. The method according to claim 15, wherein the compound represented by formula (I-1-A) is 3-chloropropionyl chloride.

18. The method according to claim 15, wherein the amount of the base after subtracting an equivalent amount neutralized with an acid addition salt of (S)-3-((3,5-dimethoxyphenyl)ethynyl)-1-(pyrrolidin-3-yl)-1H-pyrazolo[3,4-d]pyrimidin-4-amine or a salt thereof is 0.5 to 10 equivalents per equivalent of (S)-3-((3,5-dimethoxyphenyl)ethynyl)-1-(pyrrolidin-3-yl)-1H-pyrazolo[3,4-d]pyrimidin-4-amine.

19. A method for producing (S)-1-(3-(4-amino-3-((3,5-dimethoxyphenyl)ethynyl)-1H-pyrazolo[3,4-d]pyrimidin-1-yl)pyrrolidin-1-yl)-2-propen-1-one or a pharmaceutically acceptable salt thereof, the method comprising:
producing a compound represented by formula (A-1) or a salt thereof by the method of claim 15; and
eliminating L² from the obtained compound represented by formula (A-1).

20. A method for improving filterability, the method comprising producing (S)-1-(3-(4-amino-3-((3,5-dimethoxyphenyl)ethynyl)-1H-pyrazolo[3,4-d]pyrimidin-1-yl)pyrrolidin-1-yl)-2-propen-1-one or a salt thereof by the method of claim 15.

21. A compound represented by the following formula (A-1) or a salt thereof 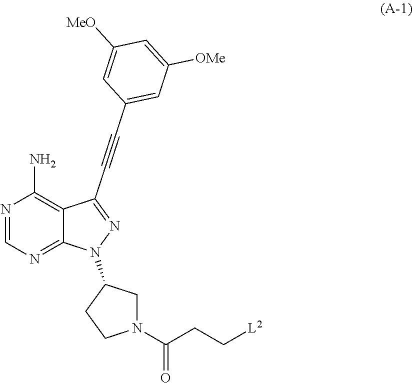
wherein L² represents a leaving group.

22. The compound or a salt thereof according to claim 21, wherein L² is a halogen atom.

23. The compound or a salt thereof according to claim 21, wherein L² is a chlorine atom.

24. The compound or a salt thereof according to claim 21 for use in determining the presence of impurities in the production of (S)-1-(3-(4-amino-3-((3,5-dimethoxyphenyl)ethynyl)-1H-pyrazolo[3,4-d]pyrimidin-1-yl)pyrrolidin-1-yl)-2-propen-1-one or a pharmaceutically acceptable salt thereof.
